(19) United States
(12) Patent Application Publication
(10) Pub. No.: US 2007/0143881 A1
(43) Pub. Date: Jun. 21, 2007

(54) Methods and Compositions for Improving the Efficiency of Site-Specific Polynucleotide Exchange

(75) Inventors: Dennis L. Bidney, Urbandale, IA (US); Sheila E. Maddock, Johnston, IA (US); Xinli E. Wu, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 11/427,947

(22) Filed: Jun. 30, 2006

Related U.S. Application Data
(60) Provisional application No. 60/751,384, filed on Dec. 16, 2005.

(51) Int. Cl. A01H 1/00

(52) U.S. Cl. 800/294

(57) ABSTRACT

Methods and compositions using a site-specific integration system are combined with methods and compositions which deliver compositions via microinjection directly to the embryo sac of a plant. The methods allow for various components of the site-specific recombination system to be introduced into the cellular environment of the embryo sac a composition comprising at least one component of the site-specific recombination system is injected into an embryo sac, providing improved efficiency of expression, recombination, integration, exchange, excision and/or inversion of a polynucleotide of interest. The polynucleotide of interest may be stably integrated into the genome of the egg cell, zygote, embryo, or endosperm, and tissues, plant parts, and/or plants produced therefrom. Cells, egg cells, zygotes, embryos, endosperm, tissues, seeds, and/or plants produced by the methods and comprising the polynucleotide(s) of interest are also provided.

### CROSS-REFERENCE TO RELATED APPLICATIONS

This application claims the benefit of U.S. Application Ser. No. 60/751,384 filed Dec. 16, 2005, which is herein incorporated by reference in its entirety.

### FIELD OF THE INVENTION

The invention relates to the field of molecular biology, more particularly to the field of plant transformation and site-specific recombination systems.

### BACKGROUND

Many plant transformation systems involve extensive callus-based culture and selection. These methodologies are time consuming and may increase the likelihood that somaclonal variants will arise that exhibit undesirable agronomic characteristics. The use of developmentally organized explants as targets for transformation minimizes tissue culture steps but increases the likelihood that chimeric plants are produced. Targeting gametes, zygotes or early stage embryos in embryo sacs for transformation is a potential solution. Although the production of gametes and zygotes by plants is well understood, reproducible methods for in vitro manipulation and transformation of these cells are needed to provide improved methods of transforming plants.

The random insertion of the introduced DNA into the genome of a host cell can be lethal if the foreign DNA happens to insert into critically important native gene. Even if a random insertion event does not impair the functioning of a native gene, the expression of an inserted foreign nucleotide sequence may be influenced by position effects caused by the surrounding genomic DNA. In some cases, the nucleotide sequence is inserted into a site where the position effect is strong enough to suppress the function or regulation of the introduced nucleotide sequence. Position effects can result in reduced agronomics of the plant, additional costs associated with further research, the creation of additional transgenic events, and slower time from trait development to product.

For these reasons, more efficient methods are needed for both transforming plant cells and for targeting the insertion, excision, inversion, or exchange of polynucleotides in the plant genome.

### SUMMARY

Methods and compositions using a site-specific integration system are combined with methods and compositions which deliver compositions via microinjection directly to the embryo sac of a plant. The methods allow for various components of the site-specific recombination system to be introduced into the cellular environment of the embryo sac. A composition comprising at least one component of the site-specific recombination system is injected into an embryo sac, providing improved efficiency of expression, recombination, integration, exchange, excision and/or inversion of a polynucleotide of interest. The polynucleotide of interest may be stably integrated into the genome of the egg cell, zygote, embryo, or endosperm, and tissues, plant parts, and/or plants produced therefrom. Cells, egg cells, zygotes, embryos, endosperm, tissues, seeds, and/or plants produced by the methods and comprising the polynucleotide(s) of interest are also provided.

### BRIEF DESCRIPTION OF DRAWINGS

FIG. 1 shows an embryo/endosperm structure derived from a 1-day post-pollinated microinjected nucellar slab after 21 days in vitro culture. The nucellar slab, isolated from a transgenic plant stably transformed with PHP10006 (Ubi::FRT1::GFP::35S::MOPAT::FRT1::GUS::FRT5), was injected with Agrobacterium containing PHP17797 (RB::Ubi-FRT1::FLPm::35S::Bar::FRT5::LB). The FLP enzyme expressed from PHP17797 mediated excision of the DNA between the FRT1 sites in PHP10006 in the injected slab, thereby creating an operable linkage of the promoterless GUS gene to the Ubi promoter, resulting in GUS expression. The blue spot (arrow) shows the GUS expression in the embryo area, demonstrating T-DNA transfer and gene expression from the injected Agrobacterium. Bar=1 mm.

### DETAILED DESCRIPTION

Methods and compositions which deliver site-specific recombination system components via microinjection into the embryo sac include, but are not limited to the following:
1. A method for targeting a polynucleotide of interest to a target site in a plant comprising
   a) providing an embryo sac from the plant, wherein said embryo sac comprises a target site comprising a first recombination site, wherein said target site is stably incorporated into the genome;
   b) injecting into said embryo sac an effective concentration of an Agrobacterium comprising a T-DNA, wherein said T-DNA comprises said first recombination site and said polynucleotide of interest, wherein said Agrobacterium is capable of T-DNA transfer into a plant cell; and,
   c) providing a recombinase, wherein said recombinase recognizes and implements recombination at the first recombination site,
whereby the polynucleotide of interest is inserted at the target site.
2. The method of 1 wherein said embryo sac of step (a) has stably incorporated into its genome the target site comprising the first recombination site and a second recombination site, wherein said first and said second recombination sites are dissimilar and non-recombinogenic with respect to one another; and, said T-DNA comprises a transfer cassette, said transfer cassette comprising, in the following order, said first recombination site, said polynucleotide of interest, and said second recombination site; and, wherein said recombinase recognizes and implements recombination at the first and the second recombination sites, whereby the polynucleotide of interest is inserted at the target site.
3. The method of 2, wherein said transfer cassette comprises, in the following order, said first recombination site, a promoter operably linked to the polynucleotide of interest, and said second recombination site.
4. The method of any one of 1-3 wherein said polynucleotide of interest comprises a selectable marker.
5. The method of 5 wherein said method further comprises monitoring expression of the selectable marker to assess promoter activity.
6. The method of 2 wherein said embryo sac of step (a) has stably incorporated into its genome a polynucleotide comprising, in the following order, a promoter operably linked to the target site comprising the first recombination site and the second recombination site, said transfer cassette comprises, in the following order, said first recombination site, said polynucleotide of interest, and said second recombination site, wherein said polynucleotide of interest is not operably linked to a promoter.
7. The method of 6 wherein said polynucleotide of interest comprises a selectable marker.
8. The method of 7, further comprising culturing said embryo sac on an appropriate selective agent to directly select the embryo sac expressing the selectable marker.
9. The method of any one of 1-8, further comprising recovering from said embryo sac a targeted plant having said polynucleotide of interest stably incorporated into its genome at the target site.
10. The method of 2, wherein said embryo sac of step (a) has stably incorporated into its genome a polynucleotide comprising, in the following order, a promoter active in said plant operably linked to an ATG translational start site operably linked to the target site comprising the first recombination site and the second recombination site; and, said transfer cassette comprising, in the following order, the first recombination site, the polynucleotide of interest, and the second recombination site, wherein the ATG translation start of the polynucleotide of interest has been replaced with said first recombination site, whereby recombination with the target site results in the polynucleotide of interest being operably linked to the ATG translational start site.
11. The method of any one of 1-10, wherein said plant has stably incorporated into its genome a polynucleotide encoding said recombinase or said T-DNA comprises the polynucleotide encoding said recombinase.
12. The method of any one of 1-11, wherein said recombinase is a FLP recombinase, or a Cre recombinase.
13. The method of any one of 1-12 wherein said recombinase is encoded by a polynucleotide having plant preferred codons.
14. The method of 13, wherein said recombinase is encoded by a polynucleotide having maize preferred codons.
15. The method of 2, wherein said transfer cassette comprises, in the following order, at least the first recombination site, a polynucleotide of interest, a third recombination site, and the second recombination site, and said first, said second, and said third recombination sites are dissimilar and non-recombinogenic with respect to one another.
16. The method of 15, further comprising recovering from said embryo sac a targeted plant having said polynucleotide of interest stably integrated into its genome at the target site.
17. The method of 16, further comprising injecting into a targeted embryo sac derived from the targeted plant a second composition comprising an effective concentration of a second Agrobacterium comprising a second T-DNA comprising a second transfer cassette comprising, in the following order, at least the second recombination site, a second polynucleotide of interest, and the third recombination site; and, providing a second recombinase, wherein said second recombinase recognizes and implements recombination at the second and the third recombination sites, and the second polynucleotide of interest is inserted at the second and third recombination sites of the target site.
18. The method of 17, further comprising recovering from said targeted embryo sac a re-targeted plant having said first and said second polynucleotide of interest stably integrated into its genome at the target site.
19. The method of 2, wherein said target site comprises, in the following order, at least the first recombination site, the polynucleotide of interest, the second recombination site, and a third recombination site; wherein said first, said second, and said third recombination sites are dissimilar and non-recombinogenic with respect to one another; said transfer cassette comprising, in the following order, the second recombination site, a second polynucleotide of interest, and the third recombination site; and, providing the recombinase, wherein said recombinase recognizes and implements recombination at the recombination sites of the transfer cassette, whereby the second polynucleotide of interest is inserted at the target site.
20. The method of 19, further comprising recovering from said targeted embryo sac a re-targeted plant having said polynucleotide of interest stably integrated into its genome at the target site.
21. The method of 17, wherein at least one of said first, said second, or said first and said second recombinase comprises a FLP recombinase.
22. The method of 17, wherein said first, said second, or said first and said second recombinase comprises a Cre recombinase.
23. The method of any one of 17, 20, or 21, wherein a polynucleotide encoding at least said first, said second or said first and said second recombinase is stably incorporated into the genome of the plant, or said T-DNA comprises the polynucleotide encoding said recombinase.
24. The method of any one of 2-23, wherein at least one of said dissimilar and non-recombinogenic recombination sites is selected from the group consisting of a FRT site, and a LOX site.
25. The method of 1, wherein said first recombination site is selected from the group consisting of a FRT site, and a LOX site.
26. The method of any one of 21 or 22, wherein the first, the second, or the first and the second recombinase is encoded by a polynucleotide having maize preferred codons.
27. The method of any one of 1-26, wherein said embryo sac comprises a fertilized embryo sac.
28. The method of 27, wherein said fertilized embryo sac comprises an embryo.
29. The method of 27, wherein said fertilized embryo sac comprises a zygote.
30. The method of 27, wherein said fertilized embryo sac comprises a one-day post-pollination embryo sac.
31. The method of any one of 1-30, wherein said plant is a monocot.
32. The method of 31, wherein said monocot is maize.
33. The method of any one of 1-30, wherein said plant is a divot.
34. A method of introducing into a plant a recombinase polypeptide comprising injecting into an embryo sac of said plant a composition comprising an Agrobacterium comprising a T-DNA comprising a polynucleotide encoding the recombinase operably linked to a promoter active in the embryo sac, wherein said Agrobacterium is capable of T-DNA transfer into a plant cell.
35. The method of 34, wherein said plant has stably incorporated into its genome a nucleotide sequence comprising, in the following order, a first recombination site, a polynucleotide sequence of interest, and a second recombination site, wherein said first and said second recombination sites are recombinogenic with respect to one another and are directly repeated, whereby the recombinase recognizes and implements recombination at the first and the second recombination site, thereby excising the polynucleotide sequence of interest from the genome of said plant.
36. The method of 34, wherein said plant has stably incorporated into its genome a target site and a transfer cassette, said target site comprising a first recombination site and a second recombination site, wherein said first and said second recombination sites are dissimilar and non-recombinogenic with respect to one another; and said transfer cassette comprises, in the following order, said first recombination site, said polynucleotide of interest, and said second recombination site; wherein said recombinase recognizes and implements recombination at the first and the second recombination site, and the polynucleotide of interest is inserted at the target site.
37. A method for locating preferred integration sites within the genome of a plant comprising
   a) injecting into an embryo sac from the plant a composition comprising an effective concentration of an Agrobacterium comprising a T-DNA comprising a first recombination site and a polynucleotide of interest, wherein said Agrobacterium is capable of T-DNA transfer into a plant cell;
   b) monitoring the level of expression of the polynucleotide of interest; and,
   c) selecting the embryo sac or the plant recovered therefrom expressing the polynucleotide of interest.
38. The method of 37 wherein said T-DNA comprises a target site comprising, in the following order, the first recombination site, the polynucleotide of interest, and a second recombination site, wherein said first and said second recombination sites are dissimilar and non-recombinogenic with respect to one another.

A site-specific integration system is combined with methods and compositions for direct delivery to the embryo sac of a plant via microinjection. The methods allow for various components of the site-specific recombination system to be introduced into the cellular environment of the embryo sac, which possibly represents a cellular environment close to that in which recombination is known to occur naturally. In particular examples, a composition comprising an effective concentration of an Agrobacterium comprising a T-DNA comprising at least one component of the site-specific recombination system is injected into an embryo sac. Such methods of introduction allow for an improved efficiency of expression, recombination, integration, exchange, excision and/or inversion of a polynucleotide. In specific examples, the polynucleotide of interest is stably integrated into the genome of the egg cell, zygote, embryo, or endosperm.

Methods and compositions are provided for introducing at least one component of a site-specific integration system into a plant via microinjection. In some examples, an Agrobacterium comprising at least one site-specific recombination component is delivered to an embryo sac by microinjection. For example, methods comprising injecting into a plant embryo sac a composition comprising an effective concentration of a biologically active Agrobacterium which is capable of T-DNA transfer into a cell are provided. The methods provide an efficient means for delivering the polynucleotide, comprising a component of a site-specific recombination system, contained in the T-DNA into an unfertilized or a fertilized embryo sac, and further provide an effective method for the expression and/or integration of the polynucleotide in a plant. The polynucleotide contained in the T-DNA can comprise at least one component of a site-specific recombination system including, but not limited to, at least one recombination site, a target site, a transfer cassette, and/or a nucleotide sequence encoding a recombinase polypeptide or a variant thereof. In some examples the T-DNA may further comprise at least one polynucleotide of interest.

An embryo sac is typically an eight-nucleate female gametophyte. The embryo sac arises from the megaspore by successive mitotic divisions and comprises three antipodal cells, the egg cell, two synergids, and the central cell, which contains the two polar nuclei. The polar nuclei unite with the nucleus of a sperm cell in a triple fusion. In certain seeds, including, cereal seeds, the product of this triple fusion develops into the 3n endosperm.

An ovule is the structure in seed plants which contains the female gametophyte. The ovule is comprised of the nucellus which is surrounded by one or two integuments, and it is attached to the placenta by a stalk known as the funiculus. A nucellus is the tissue within the ovule in which the female gametophyte, the embryo sac, develops. The nucellus is the maternal tissue that is adjacent to the embryo sac.

The embryo sac employed in the methods can be unfertilized. In other methods, a fertilized embryo sac is injected. A fertilized embryo sac is an embryo sac following the fusion of a sperm cell with the egg cell and/or the fusion of a sperm cell with the central cell. Typically, a fertilized embryo sac results from a double fertilization, wherein a first sperm cell fuses with the egg cell and a second sperm cell fuses with the central cell. The injection into the fertilized egg sac can occur at any time following fertilization including at about less than 1, 1, 2, 3, 4, 5, 6, 7, 8, 9, 10, 11, 12, 13, 14, 15 or more days post-pollination.

An egg cell is a female gamete cell. A zygote is a fertilized embryo at the one-cell stage of development and begins upon fertilization of the egg cell with the sperm and concludes upon cleavage of the zygote into a two-cell embryo.

An early stage embryo encompasses all embryonic stages that begin upon fertilization of the egg cell to form the zygote and extends through the 2-cell stage, the 4-cell stage, and the 8-cell stage.

The embryo sacs employed in the methods can be obtained using a variety of methods. An isolated embryo sac is separated from a portion of the plant but continues to retain the structural integrity of the embryo sac. In specific examples, the isolated embryo sac is surrounded by the ovary cell wall and/or the nucellus. In one method, the plant embryo sac is isolated via micromanipulation, for example, the embryo sac can be isolated by serially sectioning the ovaries. The thickness of the sections varies from species to species depending on the size of the ovule, sections of less than about 150 μm, 150 μm, 160 μm, 170 μm, 180 μm, 190 μm, 200 μm, 210 μm, 220 μm, 230 μm, 240 μm, 250 μm, 275 μm, 300 μm, 325 μm, 350 μm, 375 μm, 400 μm, 450 μm, 500 μm, and up to the thickness of the ovule can be used. For example, the thickness of sections for maize typically ranges between about 200 μm to about 300 μm. The isolated ovary sections prepared by this method comprise embryo sacs surrounded by tissue from nucellus and the ovary wall. The intact embryo sacs are contained between thin layers of nucellus and are visible using a stereomicroscope. The sections containing the embryo sacs can be readily manipulated with forceps. Additional details regarding the method of isolation can be found, for example, in U.S. Pat. No. 6,300,543 and Laurie et al. (1999) In Vitro Cell. Dev. Biol. Plant 35:320-325, both of which are herein incorporated by reference. Additional methods for this isolation of embryo sacs include dissection, micromanipulation, enzymatic maceration or any combination of methods. See, for example, Wagner et al. (1989) Plant Sci 59:127-132; Kranz et al. (1993) Plant Cell 5:739; Allington et al. (1985) The Experimental Manipulation of the Ovule Tissue, Longman, N.Y., 39-51; and Theunis et al. (1991) Sex Plant Reprod 4:145-154.

Various methods for identifying fertilized plant embryo sacs can be employed. In one example, the identification of a fertilized plant embryo sac comprises contacting pollen from a plant comprising a polynucleotide encoding a visual marker operably linked to a promoter that is active in pollen or in an embryo sac with a population of unfertilized seed, each seed comprising an embryo sac. Fertilized embryos expressing the visual marker can then be identified.

Pollen refers to the male gametophyte. Any means of contacting the pollen to the unfertilized seed can be used. For example, the pollen can be applied either artificially or naturally to the ovule (stigma) of a plant of interest.

Any promoter active in the pollen or the embryo sac can be used in this method. Such promoters include, but are not limited to, constitutive promoters, promoters that are active in the central cell of the embryo sac, promoter that are active in the zygote of the embryo sac, promoters that are active in the pollen, and/or promoters that are active in the pollen tube. A female-preferential promoter refers to a promoter having transcriptional activity only, or primarily, in one or more of the cells or tissues of a female reproductive structure of a plant. Promoters active in these tissues are known. See, for example, U.S. Pat. Nos. 6,576,815, 6,452,069, Potenza et al. (2004) In Vitro Cell Dev Biol 40:1-22, and Hamilton et al. (1998) Plant Mol Bio 38:663-9.

While any visual maker can be used to identify the fertilized embryo, in one example, the visual marker is a fluorescent protein. Such fluorescent proteins include but are not limited to yellow fluorescent protein (YFP), green fluorescent protein (GFP), cyan fluorescent protein (CFP), and red fluorescent protein (RFP). In still other examples, the visual marker is encoded by a polynucleotide having maize preferred codons. In further examples, the visual marker comprises GFPm, AmCyan, DsYellow, or ZsRed. See, Wenck et al. (2003) Plant Cell Rep 22:244-251.

In one non-limiting example, the embryos can be isolated from maize ears pollinated by a transgenic line comprising a fluorescent marker expressed in pollen. The putative pollinated embryos can be identified by screening for the pollen marker. The method can be used to identify fertilized embryo sac at any time following fertilization including at about less than 1, 1, 2, 3, 4, 5, 6, 7, 8, 9, 10, 11, 12, 13, 14, 15 or more days post-pollination.

The embryo sac used can be from any plant species, including, but not limited to, monocots and dicots. Examples of plant species of interest include, but are not limited to, corn (Zea mays), Brassica sp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), leguminous plants such as guar beans, locust bean, fenugreek, garden beans, cowpea, mungbean, fava bean, lentils, and chickpea, vegetables, ornamentals, grasses, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and Cucumis species such as cucumber (C. sativus), cantalope (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

The embryo sac employed in the methods can be unfertilized. In this method, the injected embryo sac comprises an egg cell. Before, during, and/or following injection of the embryo sac, the egg cell is fertilized with sperm cell from the plant. See, for example, Kranz “In Vitro Fertilization”, Ch. 7, pp. 143-166 in Current Trends in the Embryology of Angiosperms, Bhojwani and Soh (Eds.), 2001 Kluwer Academic Publishers; and U.S. Pat. No. 6,300,543. Plants can be recovered from the in vitro fertilized and injected embryo sacs. In still other examples, the developing central cell and/or endosperm in the embryo sacs is targeted for transformation by microinjection.

A variety of bacterial strains can be used to introduce one or more site-specific recombination component and/or a polynucleotide of interest into a plant. In one method, the Agrobacterium employed harbors at least one polynucleotide of interest operably linked to a promoter active in a plant which is located between the T-DNA borders, wherein the Agrobacterium employed is capable of T-DNA transfer into a plant cell. In other methods, the Agrobacterium employed harbors a recombinase or biologically active variant thereof that is located between the T-DNA borders. In other methods, the recombinase may be located outside the T-DNA borders of the Agrobacterium used for delivery. The Agrobacterium employed in the methods contain the necessary genetic elements for T-DNA transfer into a plant cell. A number of wild-type and disarmed strains of A. tumefaciens and A. rhizogenes harboring Ti or Ri plasmids can be used. The Agrobacterium hosts typically comprise disarmed Ti and Ri plasmids that do not contain the oncogenes which cause tumorigenesis or rhizogenesis, respectfully.

Any strain of Agrobacterium can be used, so long as it is biologically active and harbors a T-DNA comprising the desired polynucleotide. A number of references review Agrobacterium-mediated transformation in monocots and dicots. See, for example, Hellens et al. (2000) Trends Plant Sci 5:446-451; Hooykaas (1989) Plant Mol. Biol. 13:327-336; Smith et al. (1995) Crop Sci 35:301-309; Chilton (1993) Proc. Natl. Acad Sci. (USA) 90:3119-3210; and Moloney et al. (1993) In: Monograph Theor. Appl. Genet., N.Y., Springer Verlag 19:148-167.

Agrobacterium strains of interest can be wild type or derivatives thereof which have alterations that increase transformation efficiency. Strains of interest include, but are not limited to, A. tumefaciens strain C58, a nopaline-type strain (Deblaere et al. (1985) Nucleic Acids Res. 13:4777-4788); octopine-type strains such as LBA4404 (Hoekema et al. (1983) Nature 303:179-180); or succinamopine-type strains e.g., EHA101 or EHA105 (Hood et al. (1986) J. Bacteriol. 168:1291-1301); A. tumefaciens strain A281 (U.S. Patent Publication No. 20020178463); GV2260 (McBride et al. (1990) Plant Mol. Biol. 14:269-276); GV3100 or GV3101 (Holsters et al. (1980) Plasmid 3:212-230); A136 (Watson et al. (1975) J. Bacteriol. 123:255-264); GV3850 (Zambryski et al. (1983) EMBO J 2:2143-2150); GV3101::Pmp90 (Koncz et al. (1986) Mol. Gen. Genet 204:383-396); and, AGL-1 (Lazo et al. (1991) Biotechnology 9:963-967). The methods and uses of these strains for plant transformation have been reported.

Transfer DNA or T-DNA comprises a genetic element that is capable of integrating a polynucleotide contained within its borders into another polynucleotide. The T-DNA can comprise the entire T-DNA, but need only comprise the minimal sequence necessary for cis transfer, typically the right or left border is sufficient. The T-DNA can be synthetically derived or can be from an A. rhizogene Ri plasmid or from an A. tumefaciens Ti plasmid, or functional derivatives thereof. Any polynucleotide to be transferred, for example a recombinase, a polynucleotide of interest, a recombination site, a restriction site, a recognition site, a sequence tag, a target site, a transfer cassette and/or, optionally, a marker sequence may be positioned between the left border sequence and the right border sequence of the T-DNA. The sequences of the left and right border sequences may or may not be identical and may or may not be inverted repeats of one another. It is also possible to use only one border, or more than two borders, to accomplish transfer of a desired polynucleotide.

Various plasmids are available comprising T-DNAs that can be employed in the methods. For example, many Agrobacterium employed for the transformation of dicotyledonous plant cells contain a vector having a DNA region originating from the virulence (vir) region of the Ti plasmid. The Ti plasmid originated from A. tumefaciens, and a polynucleotide of interest and/or the sequence encoding the recombinase can be inserted into this vector. Alternatively, the polynucleotide of interest and/or sequence encoding the recombinase can be contained in a separate plasmid which is then inserted into the Ti plasmid in vivo, in Agrobacterium, by homologous recombination or other equivalently resulting processes. See, for example, Herrera-Esterella et al. (1983) EMBO J. 2:987-995 and Horch et al. (1984) Science 223:496-498.

A vector has also been developed which contains a DNA region originating from the virulence (vir) region of Ti plasmid pTiBo542 (Jin et al. (1987) J. Bacteriol. 169:4417-4425) contained in a super-virulent A. tumefaciens strain A281 showing extremely high transformation efficiency. This particular vector includes regions that permit vector replication in both E. coli and Agrobacterium. See, Hood et al. (1984) Bio/Tech 2:702-709; Komari et al. (1986) Bacteriol 166:88-94. This type of vector is known as a superbinary vector (see European Patent Application 0604662A1). Examples of superbinary vectors include pTOK162 and pTIBo542 (U.S. Patent Publication No. 2002178463 and in Japanese Laid-Open Patent Application no. 4-222527); pTOK23 (Komari et al. (1990) Plant Cell Rep. 9:303-306); pPHP10525 (U.S. Pat. No. 6,822,144). See, also Ishida et al. (1996) Nat Biotech. 14:745-750.

Additional transformation vectors comprising T-DNAs that can be used include, but are not limited to, pBIN19 (Bevan et al. (1984) Nucleic Acids Res 12:8711-8721); pC22 (Simoens et al. (Nucleic Acids Res 14:8073-8090); pGA482 (An et al. (1985) EMBO J 4:277-284); pPCV001 (Koncz et al. (1986) Mol. Gen. Genet. 204:383-396); pCGN1547 (McBride et al. (1990) Plant Mol. Biol. 14:269-276); pJJ1881 (Jones et al. (1992) Transgenic Res. 1:285-297); pPzP111 (Hajukiewicz et al. (1994) Plant Mol. Biol. 25:989-994); and, pGreen0029 (Hellens et al. (2000) Plant Mol. Biol. 42:819-832).

The polynucleotide may be inserted into a restriction site in the T-DNA region of the transformation vector, and the desired recombinant vector may be selected depending on an appropriate selection marker, such as drug resistance and the like contained on the plasmid employed. General molecular biological techniques used are provided, for example, by Sambrook et al. (eds.) (1989) Molecular Cloning: A Laboratory Manual, Cold Spring Harbor Laboratory Press, Cold Spring Harbor, N.Y.

The introduction of a vector into a bacterium belonging to genus Agrobacterium such as A. tumefaciens can be carried out by various conventional methods including, the triple cross method (Ditta (1980) Proc. Natl. Acad. Sci. USA 77:7347-7351) and the methods disclosed in Ishida et al. (1996) Nat Biotech 14:745-750 and in U.S. Patent Publication No. 20010054186. The Agrobacterium having the desired transformation plasmid can be isolated based on the use of a selectable marker incorporated into the vector.

Methods to determine if an Agrobacterium is capable of T-DNA transfer into a plant cell are known. For example, Agrobacterium harboring the vector carrying the T-DNA comprising an appropriate marker is contacted to the appropriate plant tissue. Agrobacterium capable of T-DNA transfer can be identified by their ability to transfer the T-DNA, and the accompanying marker, into the plant tissue. Methods to assay for various markers are routine. See, for example, Shurvinton et al. (1992) Proc. Natl. Acad. Sci. 89:11837-11841; Mysore et al. (1998) Mol. Plant Microb Interact. 11:668-683; DeNeve et al. (1997) Plant J. 11:15-29; and, DeBuck et al. (1998) Mol. Plant Microb Interact. 11:449-57.

The composition comprising the Agrobacterium that is injected into the embryo sac can further comprise one or more surfactants. In one example, a surfactant is added to the Agrobacterium suspension to enhance the number of embryos obtained expressing the polynucleotide of interest. A surfactant or surface-active agent refers to any compound that can reduce surface tension when dissolved in water or water solutions or that can reduce interfacial tension between a liquid (e.g., water) and a solid (e.g., bacteria). Generally, the surfactant should not be harmful to the plant, the bacterium, or any other component of the injection composition. Surfactants may be categorized as anionic, nonionic, zwitterionic, or cationic, depending on whether they comprise one or more charged groups. Anionic surfactants contain a negatively charged group and have a net negative charge. Nonionic surfactants contain non-charged polar groups and have no charge.

Suitable surfactants that can be used in the aqueous medium can include Triton™ brand of surfactants, the Tween™ brand of surfactants and the Silwet™ brand of surfactants. The Triton™ brand of surfactants includes specialty surfactants that are alcohols and ethoxylates, alkoxylates, sulfates, sulfonates, sulfonosuccinates or phosphate esters. One widely used non-ionic surfactant is Triton™ X-100 (t-Octylphenoxypolyethoxyethanol). Another surfactant is Silwet-L77® (polyalkyleneoxide modified heptamethyltrisiloxane). The concentration of surfactants used will vary with the type of surfactant and plant being used. Generally, surfactants are used in concentrations ranging from 0.005% to about 1% of the volume of the Agrobacterium suspension. Concentrations can also range from 0.005% to about 0.5% or from about 0.005% to about 0.05%. In some examples a combination of compatible surfactants can be used.

Any composition of interest can be microinjected into the embryo sac, including but not limited to, DNAs (naked or coated), RNAs, mRNAs, miRNAs, oligos, siRNAs, proteins, peptides, conjugated dyes (FITC-Dextran), dyes, viral vectors, replicons, amplicons, DNA/RNA chimeras, carbohydrates, cofactors, protein complexes, any site-specific integration component, Agrobacterium, or any combination thereof. In addition, any of the various components listed above can be provided to the embryo sac using any method known for introduction, including, but not limited to, microinjection into the embryo sac, or particle bombardment, agroinfection, etc.

In some examples, the methods introduce a desired polynucleotide into a plant via microinjection of an Agrobacterium directly into an embryo sac. The polynucleotide can be either stably integrated into the genome or be transiently expressed. Stable transformation indicates that the desired polynucleotide integrates into the genome of the plant and is capable of being inherited by the progeny thereof. Transient transformation indicates that a sequence composition is introduced into the plant and is present and/or expressed in the plant for a limited period of time.

Techniques for microinjection of various substances into a cell of interest are known. Generally, such injections occur by means of a glass microcapillary-injection pipette and employ the use of a micromanipulator (Crossway (1986) Mol. Gen. Genet. 202:179-185 and Morikawa et al. (1985) Plant Cell Physiol. 26:229-236).

Procedures for growth and suitable culture conditions for Agrobacterium as well as subsequent inoculation procedures are known. The density of the Agrobacterium culture used for microinjection can vary from one system to the next, and therefore optimization of these parameters may be required. An effective concentration of the bacterium inoculum comprises a concentration of Agrobacterium which, when injected into the embryo sac of a plant, is sufficient to allow for the recovery of a plant expressing the desired polynucleotide and/or allow for the desired polynucleotide to be integrated at the target site. Such concentration ranges include, for example, an Agrobacterium optical density (OD₅₅₀) of about 0.5, 0.1, 0.05, 0.01, 0.005, 0.001, or 0.0005. In some examples, the effective concentration of Agrobacterium in the inoculum is about 0.1 O.D. In other examples, the O.D. of the Agrobacterium inoculum is about 0.5 to about 0.1, about 0.1 to about 0.05, about 0.05 to about 0.01, about 0.01 to about 0.005, about 0.005 to about 0.001, or about 0.001 to about 0.0005. The concentration of the Agrobacterium in the inoculum is only limited by the highest level of bacterium that does not clog the micropipette and the lowest level of bacterium which can produce bacterial growth when dispensed in a microinjection droplet onto standard bacterial growth media. In addition, the effective concentration of Agrobacterium will be sufficient to produce the desired number of integration events and produce an acceptable integration pattern for the desired purpose.

In some examples, a dye marker can be used with the injection composition in order to confirm delivery to the nucellar slab and/or embryo sac. Typically, the dye used is a vital dye, and may be conjugated to an inert matrix. Any suitable dye, compatible with the injection parameters, Agrobacterium, and cell culture can be used. For example, a fluorescent marker such as FITC-Dextran can be co-injected with the Agrobacterium, and delivery confirmed using a fluorescence microscope.

A variety of tissue culture media, which when supplemented appropriately, support plant tissue growth and development and are suitable for plant transformation and regeneration. These tissue culture media can either be purchased as a commercial preparation or custom prepared and modified. Examples of such media include, but are not limited to, Murashige and Skoog (Murashige et al. (1962) Physiol. Plant 15:473-497), N6 (Chu et al. (1975) Scienta Sinica 18:659), Linsmaier and Skoog (Linsmaier et al. (1965) Physio. Plant 18:100), Uchimiya and Murashige (Uchimiya et al. (1974) Plant Physiol. 15:473), Gamborg's media (Gamborg et al. (1968) Exp. Cell Res. 50:151), D medium (Duncan et al. (1985) Planta 165:322-332), McCown's Woody plant media (McCown et al. (1981) HortScience 16:453), Nitsch and Nitsch (Nitsch et al. (1969) Science 163:85-87), and Schenk and Hildebrandt (Schenk et al. (1972) Can. J. Bot. 50:199-204) or derivations of these media supplemented accordingly. Media and media supplements, such as nutrients and growth regulators for use in transformation and regeneration and other culture conditions such as light intensity during incubation, pH, and incubation temperatures, can be optimized for the particular target plant of interest.

The methods and examples employ at least one component from at least one site-specific recombination system. Site-specific recombinase systems, recombinases, recombinase sites and various uses are disclosed in WO99/25821, WO99/25854, WO99/25840, WO99/25855, WO99/25853, and WO 01/23545, all of which are herein incorporated by reference. A recombinase is a polypeptide that catalyzes conservative site-specific recombination between its compatible recombination sites. For reviews of site-specific recombinases, see Sauer (1994) Curr Op Biotech 5:521-527 and Sadowski (1993) FASEB 7:760-767. The recombinase used in the methods can be a naturally occurring full-length recombinase, or a biologically active fragment or a variant of the recombinase polypeptide and/or polynucleotide. Useful recombinases include recombinases from the Integrase and Resolvase families, biologically active variants and fragments thereof, and any other naturally occurring or recombinantly produced enzyme or variant thereof, that catalyzes conservative site-specific recombination between specified DNA recombination sites.

The Integrase family of recombinases has over one hundred members and includes, for example, FLP, Cre, lnt, and R. For other members of the Integrase family, see for example, Esposito et al. (1997) Nucleic Acids Res 25:3605-3614 and Abremski et al. (1992) Protein Engineering 5:87-91. Other recombination systems include, for example, the streptomycete bacteriophage phi C31 (Kuhstoss et al. (1991) J. Mol. Biol. 20:897-908); the SSV1 site-specific recombination system from Sulfolobus shibatae (Maskhelishvili et al. (1993) Mol. Gen. Genet. 237:334-342); and a retroviral integrase-based integration system (Tanaka et al. (1998) Gene 17:67-76). In some examples, the recombinase does not require cofactors or a supercoiled substrate. Such recombinases include Cre, FLP, and active variants or fragments thereof (see, e.g., WO 99/25840 and WO 99/25841, herein incorporated by reference).

FLP recombinase catalyzes a site-specific reaction involved in amplifying the copy number of the two-micron plasmid of S. cerevisiae during DNA replication. FLP recombinase catalyzes site-specific recombination between two FRT sites. The FLP protein has been cloned and expressed. See, for example, Cox (1993) Proc. Natl. Acad. Sci. U.S.A. 80:4223-4227. The FLP recombinase for use in the methods and with the compositions may be derived from the genus Saccharomyces. One can also synthesize a polynucleotide comprising the recombinase using plant-preferred codons. A FLP enzyme encoded by a nucleotide sequence comprising maize preferred codons (FLPm) that catalyzes site-specific recombination events is known. See, for example, U.S. Pat. No. 5,929,301. Additional functional variants and fragments of FLP are known. See, for example, Buchholz et al. (1998) Nat. Biotechnol. 16:617-618, Hartung et al. (1998) J. Biol. Chem. 273:22884-22891, Saxena et al. (1997) Biochim Biophys Acta 1340(2):187-204, and Hartley et al. (1980) Nature 286:860-864.

The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; Shaikh et al. (1977) J. Biol. Chem. 272:5695-5702; and, Buchholz et al. (1998) Nat. Biotechnol. 16:617-618. The Cre polynucleotide sequences may also be synthesized using plant-preferred codons. Such sequences (moCre) are described in WO 99/25840.

A chimeric recombinase can be used and comprises a recombinant fusion protein which is capable of catalyzing site-specific recombination between recombination sites that originate from different recombination systems. That is, if a set of recombination sites, characterized as being dissimilar and non-recombinogenic with respect to one another, is utilized in the methods and compositions, and the set comprises a FRT site and a Lox site, a chimeric FLP/Cre recombinase can be used. Optionally, variants, fragments, and/or both recombinases may be separately provided. Methods for the production and use of such chimeric recombinases or active variants or fragments thereof are described in WO 99/25840.

Fragments and variants of the polynucleotides encoding recombinases and fragments and variants of the recombinase proteins can be used. A fragment is a portion of the polynucleotide, and hence the protein encoded thereby, or a portion of the polypeptide. Fragments of a polynucleotide may encode protein fragments that retain the biological activity, for example, and active fragment of a recombinase implements a recombination event. Fragments of a polynucleotide may range from at least about 20 nucleotides, about 50 nucleotides, about 100 nucleotides, and up to the full-length polynucleotide.

A fragment of a polynucleotide that encodes a biologically active portion of a recombinase protein typically encodes at least 15, 25, 30, 50, 100, 150, 200, or 250 contiguous amino acids, or up to the total number of amino acids present in a full-length recombinase protein.

A biologically active portion of a recombinase protein can be prepared by isolating a portion of one of the polynucleotides encoding the portion of the recombinase polypeptide, expressing the encoded portion of the recombinase protein, and assessing the activity. Polynucleotides that encode fragments of a recombinase polypeptide can comprise a nucleotide sequence comprising at least 16, 20, 50, 75, 100, 150, 200, 250, 300, 350, 400, 450, 500, 550, 600, 650, 700, 800, 900, 1,000, 1,100, 1,200, 1,300, or 1,400 nucleotides, or up to the number of nucleotides in a full-length recombinase nucleotide sequence disclosed herein.

Variant sequences have a high degree of sequence similarity. For polynucleotides, conservative variants include those sequences that, because of the degeneracy of the genetic code, encode the amino acid sequence of one of the recombinase polypeptides. Variant polynucleotides can be identified and/or isolated by standard techniques, for example, polymerase chain reaction (PCR) and hybridization techniques. Variant polynucleotides also include synthetically derived nucleotide sequences, for example generated using site-directed mutagenesis, which still encode a recombinase protein. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the original polynucleotide.

Variants of a particular polynucleotide can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

A variant protein is a protein derived from a native protein by deletion, addition, or substitution of one or more amino acids at the N-terminal, C-terminal, and/or an internal site in the native protein. Variant proteins retain the biological activity of the native protein, for example a variant recombinase implements a recombination event between appropriate recombination sites. Such variants may result from, for example, genetic polymorphism or from human manipulation. Biologically active variants of a recombinase protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters. A biologically active variant of a protein may differ from that protein by as few as 1-15 amino acid residues, as few as 1-10, such as 6-10, as few as 5, as few as 4, 3, 2, or even 1 amino acid residue.

Proteins may be altered in various ways including amino acid substitutions, deletions, truncations, and insertions. Methods for such manipulations are generally known. For example, amino acid sequence variants of recombinase proteins can be prepared by mutations in the DNA. Methods for mutagenesis and nucleotide sequence alterations are known. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that are not expected to affect biological activity of the protein may be found in the model of Dayhoff et al. (1978) Atlas of Protein Sequence and Structure (Natl. Biomed. Res. Found., Washington, D.C.). Conservative substitutions include exchanging one amino acid with another having similar properties.

The recombinase polynucleotides and/or polypeptides used include naturally occurring sequences, as well as modified forms. Such variants will continue to possess the ability to implement a recombination event. Generally, the modifications made in the polynucleotide encoding the variant polypeptide should not place the sequence out of reading frame, and/or create complementary regions that could produce secondary mRNA structure. See, EP Patent Application Publication No. 75,444.

Deletions, insertions, and substitutions of the protein sequences are not expected to produce radical changes in the characteristics of the protein. The exact effect of the substitution, deletion, or insertion can be evaluated by routine screening assays. Assays for recombinase activity are known and generally measure the overall activity of the enzyme on DNA substrates containing recombination sites. For example, to assay for FLP activity, inversion of a DNA sequence in a circular plasmid containing two inverted FRT sites can be detected as a change in position of restriction enzyme sites; Vetter et al. (1983) PNAS 80:7284. Alternatively, excision of DNA from a linear molecule or intermolecular recombination frequency induced by the enzyme may be assayed, as described, for example, in Babineau et al. (1985) J Biol Chem 260:12313; Meyer-Leon et al. (1987) Nucleic Acids Res 15:6469; and Gronostajski et al. (1985) J Biol Chem 260:12328. Alternatively, recombinase activity may also be assayed by excision of a sequence flanked by recombinogenic recombination sites to activate an assayable marker gene.

Variant polynucleotides and proteins also encompass sequences and proteins derived from a mutagenic and recombinogenic procedure such as DNA shuffling. With such a procedure, one or more different recombinase coding sequences can be manipulated to create a new recombinase protein possessing the desired properties. Generally, libraries of recombinant polynucleotides are generated from a population of related polynucleotides comprising sequence regions having substantial sequence identity and can be homologously recombined in vitro or in vivo. Strategies for DNA shuffling are known. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The methods and compositions further employ recombination sites. A recombination site is a polynucleotide that is recognized by the recombinase polypeptide of interest, and which is recombinogenic with a corresponding recombination site in the presence of an appropriate recombinase. Recombination sites include naturally occurring sequences, as well as fragments and variants that retain biological function. As outlined above, many recombination systems are known and one of skill will recognize the appropriate recombination site to be used with the recombination system of interest. Biologically active variants and/or fragments of recombination sites can be used in the various methods and/or compositions. Assays for recombination site activity are known, and are similar to assays for recombinase activity. For example, recombination activity can be measured by inversion of a DNA sequence in a circular plasmid containing two inverted recombination sites as a change in position of restriction enzyme sites (Vetter et al. (1983) PNAS 80:7284). Alternatively, excision of DNA flanked by directly repeated recombination sites from a linear molecule or intermolecular recombination frequency induced by the recombinase may be assayed, as described, for example, in Babineau et al. (1985) J Biol Chem 260:12313; Meyer-Leon et al. (1987) Nucleic Acids Res 15:6469; and Gronostajski et al. (1985) J Biol Chem 260:12328. Examples of recombination sites include, but are not limited to, FRT sites including, for example, the full-length native FRT site, the minimal native FRT site, functional variants of FRT, including but not limited to, FRT5, FRT6, FRT7, FRT87, and the other functional modified FRT sites. See, for example, WO 99/25821, WO 01/23545, and U.S. Provisional Application No. 60/700,225, filed on Jul. 18, 2005, all of which are herein incorporated by reference.

Recombination sites from the Cre/Lox site-specific recombination system can also be used. Such recombination sites include, for example, native LOX sites and various functional variants of LOX (see, for example U.S. Pat. No. 6,465,254, and WO 01/11058). An analysis of the recombination activity of variant LOX sites is presented in Lee et al. (1998) Gene 216:55-65 and in U.S. Pat. No. 6,465,254. Also, see for example, Schlake and Bode (1994) Biochemistry 33:12746-12751; Huang et al. (1991) Nucleic Acids Res 19:443-448; Sadowski (1995) In Progress in Nucleic Acid Research and Molecular Biology Vol. 51, pp. 53-91; Cox (1989) In Mobile DNA, Berg and Howe (eds) American Society of Microbiology, Washington D.C., pp. 116-670; Dixon et al. (1995) Mol. Microbiol. 18:449-458; Umlauf and Cox (1988) EMBO 7:1845-1852; Buchholz et al. (1996) Nucleic Acids Res24:3118-3119; Kilby et al. (1993) Trends Genet. 9:413-421; Rossant and Geagy (1995) Nat. Med. 1: 592-594; Albert et al. (1995) Plant J. 7:649-659; Bayley et al. (1992) Plant Mol. Biol. 18:353-361; Odell et al. (1990) Mol. Gen. Genet. 223:369-378; Dale and Ow (1991) Proc. Natl. Acad. Sci. USA 88:10558-10562; Qui et al. (1994) Proc. Natl. Acad. Sci. USA 91:1706-1710; Stuurman et al. (1996) Plant Mol. Biol. 32:901-913; Dale et al. (1990) Gene 91:79-85; Albert et al. (1995) Plant J. 7:649-659; and WO 01/00158.

Other integrase systems can also be used, such as those from lambda and phiC31. The recombinases, and variants thereof, as well as the recombination sites and variants thereof are known, see for example WO 03/083045, WO 02/08409, U.S. Pat. No. 5,888,732, and Lorbach et al. (2000) J Mol Biol 296:1175-1181.

Sets of dissimilar recombination sites can be used in the various methods. Accordingly, any suitable recombination site or set of recombination sites may be utilized, including a FRT site, a functional variant of a FRT site, a LOX site, and functional variant of a LOX site, an attB, attP, attL, attR site and any functional variants, any combination thereof, or any other combination of recombination sites.

The target site and transfer cassette comprise at least one recombination site. The site-specific recombinase that is used will depend upon the recombination sites present in the target site and the transfer cassette. That is, if FRT sites are utilized, a FLP recombinase, or active variant or fragment thereof will be used. In the same manner, where Lox sites are utilized, a Cre recombinase, or active variant or fragment thereof is used. If att sites are used, an lnt recombinase will be used. For example, if the set of functional recombination sites comprises both a FRT site and a Lox site, either a chimeric FLP/Cre recombinase, both FLP and Cre recombinases, or active variants and/or fragments of a recombinase or chimeric recombinase will be provided.

In addition, the recombination sites employed in the methods can be corresponding sites or dissimilar sites. Corresponding recombination sites, or a set of corresponding recombination sites, comprise recombination sites having the same nucleotide sequence.

In other examples, the recombination sites are dissimilar. Dissimilar recombination sites, or a set of dissimilar recombination sites, are recombination sites having distinct sequences comprising at least one nucleotide difference. The recombination sites within a set of dissimilar recombination sites can be either recombinogenic or non-recombinogenic with respect to one another.

Recombinogenic indicates a set of recombination sites capable of recombining with one another, and includes corresponding and dissimilar sites. Unless otherwise stated, recombinogenic recombination sites or a set of recombinogenic recombination sites include those sites where the relative excision efficiency of recombination between the sites is about 2%, 5%, 10%, 20%, 30%, 40%, 50%, 75%, 100%, or greater. The relative recombination excision efficiency is the excision efficiency in the presence of the native recombinase of a first modified recombination site with a second recombination site divided by the excision efficiency of a pair of the appropriate native recombination sites. For example, for modified FRT sites, the relative recombination excision efficiency is defined as the excision efficiency in the presence of native FLP of a first modified FRT site with a second FRT site divided by the excision efficiency of a pair of native FRT sites.

Non-recombinogenic are a set of recombination sites which, in the presence of the appropriate recombinase, will not recombine with one another, or recombination between the sites is minimal. Unless otherwise stated, non-recombinogenic recombination sites or a set of non-recombinogenic recombination sites include those sites where the relative excision efficiency of recombination between the sites is lower than 2%, 1.5%, 1%, 0.75%, 0.5%, 0.25%, 0.1%, 0.075, 0.005%, or 0.001%. For example wild type FRT1, and mutant FRT5 are non-recombinogenic. Any suitable set of non-recombinogenic or recombinogenic recombination sites may be utilized, including a FRT site or variant thereof, a LOX site or variant thereof, an att site or variant thereof, any combination of recombinogenic and/or non-recombinogenic FRT sites, LOX sites, or variants thereof, or any other combination of other recombinogenic or non-recombinogenic recombination sites known.

Sequence relationships can be analyzed and described using computer-implemented algorithms. The sequence relationship between two or more polynucleotides, or two or more polypeptides can be determined by determining the best alignment of the sequences, and scoring the matches and the gaps in the alignment, which yields the percent sequence identity, and the percent sequence similarity. Polynucleotide relationships can also be described based on a comparison of the polypeptides each encodes. Many programs and algorithms for the comparison and analysis of sequences are known.

Unless otherwise stated, sequence identity/similarity values provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff and Henikoff (1989) Proc. Natl. Acad. Sci USA 89:10915). GAP uses the algorithm of Needleman and Wunsch (1970) J. Mol. Biol. 48:443-453, to find the alignment of two complete sequences that maximizes the number of matches and minimizes the number of gaps.

The methods can employ target sites and transfer cassettes to manipulate, exchange, excise, alter, invert, and/or introduce a nucleotide sequence in vivo or in vitro. A target site is a polynucleotide that is immediately flanked by at least one recombination site. In some examples, the target site comprises at least two recombination sites, for example, sets of functional recombination sites that are dissimilar and non-recombinogenic with respect to one another; corresponding and recombinogenic with respect to one another; or dissimilar and recombinogenic with respect to one another. One or more intervening sequences may be present between the recombination sites of the target site. Intervening sequences include linkers, adapters, restriction sites, selectable markers, nucleotide sequences of interest, promoters, and/or other sites that aid in vector construction or analysis. It is further recognized that a recombination site can be contained within a nucleotide sequence of interest, for example, in introns, coding sequence, and/or 5′ and 3′ regulatory regions.

It is recognized that the plant/embryo sac may comprise multiple target sites. Multiple independent manipulations of each target site in the plant/embryo sac are available. Additionally, the target site may also comprise an expression cassette comprising a nucleotide sequence encoding an appropriate recombinase.

The methods further employ transfer cassettes. A transfer cassette is a polynucleotide that is flanked by at least a first recombination site, wherein the first site-corresponds to a recombination site in the target site. The recombination site in the transfer cassette may be identical or dissimilar to a recombination site in the target site, provided that at least one recombination site in the transfer cassette is recombinogenic with a recombination site at the target site. In some examples, the transfer cassette comprises at least a first recombination site and a second recombination site, wherein at least one of the first and/or second recombination sites correspond to a recombination site in the target site. The first and the second functional recombination sites of the transfer cassette can be dissimilar and non-recombinogenic with respect to one another. The nucleotide sequence between the recombination sites of the target site will be exchanged with the nucleotide sequence between the recombination sites of the transfer cassette. Flanked by refers to a position immediately adjacent to the sequence.

The transfer cassette can further comprise a polynucleotide of interest. The recombination sites may be directly contiguous with the polynucleotide of interest or there may be one or more intervening sequences present between one or both ends of the polynucleotide of interest and the recombination sites. Intervening sequences include linkers, adapters, restriction sites, selectable markers, additional polynucleotides of interest, promoters and/or other sites that aid in vector construction or analysis. It is further recognized that a recombination sites can be contained within the polynucleotide of interest, for example, within introns, coding sequence, and/or 5′ and 3′ untranslated regions.

Any means can be used to bring together the various components of the recombination system. A variety of methods are available for the introduction of nucleotide sequences and polypeptides into a plant, including, for example, transformation, sexual crossing, the introduction of the polypeptide, DNA, or mRNA into the cell, and/or introduction of the polynucleotide via injection of Agrobacterium. See, also, WO99/25884 and U.S. Provisional Application No. 60/751,385, entitled “Methods for Introducing Into a Plant a Polynucleotide of Interest”, filed concurrently herewith.

Providing includes any method that allows for an amino acid sequence and/or a polynucleotide to be brought together with the recited components. For instance, a cell can be provided with one or more of these various components via a variety of methods including transient and stable transformation methods or injection of Agrobacterium comprising a component of the site-specific integration system directly into the embryo sac. In other methods, the recombinase can be provided by co-injection of a recombinase DNA, mRNA or protein directly into the embryo sac. Alternatively, the plant line employed for the initial transformation of the transfer cassette can express the recombinase via a constitutive, inducible, developmental/temporal, or spatially regulated promoter, etc. In still other methods, the embryo sac can be injected with an Agrobacterium carrying a polynucleotide encoding a recombinase or biologically active variant or fragment thereof.

Following microinjection, the injected embryo sacs are cultured on appropriate media. In some examples, the injected embryo sacs are initially cultured in media that contains no selection agent. Such culture conditions are referred to as a resting phase. The duration of the resting phase can vary, depending on the nature of the plant system being injected and the nature of the marker being used. In some examples, the resting phase ranges from about less than one hour to about 72 hours, for example from about 6 to 36 hours. Depending on the system employed, injected embryos having the desired polynucleotide introduced can be selected in the presence or absence of selection pressure. See, for example, U.S. Provisional Application No. 60/751,385, entitled “A Method For Introducing Into a Plant a Polynucleotide of Interest” filed concurrently herewith, and herein incorporated by reference.

The transformants produced are subsequently analyzed to determine the presence or absence of a particular polynucleotide. Molecular analyses can include, but are not limited to Southern blots, PCR (polymerase chain reaction) analyses, immunodiagnostic approaches, and/or field evaluations. These and other methods can be performed to confirm the stability of the transformed plants produced by the methods disclosed.

The embryo sacs that have been transformed may be grown into plants in conventional ways. See, for example, McCormick et al. (1986) Plant Cell Reports 5:81-84, Laurie et al. (1999) In Vitro Cell. Dev. Biol. Plant 35:320-325, and U.S. Pat. No. 6,300,543. These plants may then be grown, and either self-pollinated, backcrossed, or outcrossed, and progeny having integration and/or expression of the desired polynucleotide identified. Two or more generations may be grown to ensure that the polynucleotide is stably maintained and inherited by the progeny, and then seeds harvested. In this manner, transformed seed having the polynucleotide of interest stably incorporated into their genome are provided.

A plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants comprising the introduced polynucleotide are also included.

A method for targeting the insertion of a polynucleotide of interest to an integration site in a plant is provided. In one example, the method comprises providing an embryo sac from the plant, wherein the plant has stably incorporated into its genome a first recombination site, and injecting into the embryo sac a composition comprising an effective concentration of an Agrobacterium comprising a T-DNA. The T-DNA comprises the first recombination site or a variant of the first recombination site and the polynucleotide of interest, wherein the Agrobacterium is capable of T-DNA transfer into a plant cell and the variant of the first recombination site is dissimilar and recombinogenic with respect to the first recombination site. A recombinase, or a biologically active variant of the recombinase, is provided wherein the recombinase or biologically active variant thereof recognizes and implements recombination at the first recombination site, and the polynucleotide of interest is inserted at the first recombination site.

In specific methods, a targeted plant having the polynucleotide of interest stably integrated into its genome is recovered from the embryo sac. A targeted plant, seed or embryo sac has stably incorporated into its genome a DNA construct that has been manipulated and/or generated through the use of a site-specific recombination system. The recombination systems employed can be used to introduce an unlimited number of changes into a plant or embryo sac. A re-targeted seed, plant, or embryo sac has undergone at least two manipulations of its genome by a site-specific recombination system.

In another example, the method comprises providing an embryo sac from the plant, wherein the plant has stably incorporated into its genome a target site comprising a first recombination site and a second recombination site, where the first and the second recombination sites are dissimilar and non-recombinogenic with respect to one another. The embryo sac is injected with a composition comprising an effective concentration of an Agrobacterium comprising a T-DNA comprising a transfer cassette. The transfer cassette comprises in the following order the first recombination site, a polynucleotide of interest, and a second recombination site, wherein the Agrobacterium is capable of T-DNA transfer into a plant cell. A recombinase or a biologically active variant of the recombinase is provided, and the recombinase or biologically active variant thereof recognizes and implements recombination at the first and the second recombination site, and the polynucleotide of interest is inserted at the target site. In some examples, the target site comprises a polynucleotide of interest. In this example, the sequence of interest in the target site is exchanged for a second polynucleotide of interest contained in the transfer cassette.

In other examples, the compositions can be used in methods to reduce the complexity of integration of transgenes in the genome of a plant. In this method, plants having simple integration patterns in their genomes are selected. A simple integration pattern indicates that the transfer cassette integrates predominantly at the target site, and at less than about 25, 20, 15, 10, 9, 8, 7, 6, 5, 4, 3, 2, or 1 random positions in the genome. Methods for determining the integration patterns are known and include, for example, Southern blot analysis and RFLP analysis.

A method to directly select a transformed plant is also provided. The method comprises providing an embryo sac from a plant having stably incorporated into its genome a polynucleotide comprising, in the following order, a promoter operably linked to the target site comprising the first recombination site and the second recombination site. The embryo sacs are injected with an effective concentration of an Agrobacterium comprising a T-DNA comprising a transfer cassette. The transfer cassette comprises in the following order the first recombination site, a polynucleotide of interest comprising a selectable marker wherein the polynucleotide of interest is not operably linked to a promoter, and the second recombination site, wherein the Agrobacterium is capable of T-DNA transfer into a plant cell. A recombinase is provided that recognizes and implements recombination at the first and second recombination sites, and thereby operably linking the selectable marker to the promoter. The embryo sac is then grown on the appropriate selective agent, or otherwise screened, to recover an embryo sac or a plant that is expressing the selectable marker and has undergone targeted integration of the transfer cassette at the target site. In some examples, a biologically active variant or fragment of the recombinase is provided.

The activity of various promoters at a characterized location in the genome of a plant can also be determined. Thus, the desired activity and/or expression level of a polynucleotide sequence of interest can be achieved, as well as, the characterization of promoters for expression in the plant. In one example, a method for assessing promoter activity in the plant comprises providing an embryo sac from the plant, wherein the plant has stably incorporated into its genome a target site comprising a first recombination site and a second recombination site, wherein the first and the second recombination sites are dissimilar and non-recombinogenic with respect to one another. The embryo sac is injected with a composition comprising an effective concentration of an Agrobacterium comprising a T-DNA comprising a transfer cassette, wherein the transfer cassette comprises, in the following order, the first recombination site, a promoter operably linked to the polynucleotide of interest comprising a selectable marker, and the second recombination site, wherein the Agrobacterium is capable of T-DNA transfer into a plant cell. A recombinase is provided wherein the recombinase recognizes and implements recombination at the first and the second recombination site, and the polynucleotide of interest is inserted at the target site. A targeted plant is recovered from the embryo sac having the polynucleotide of interest stably integrated into its genome, and expression of the selectable marker is monitored to assess promoter activity. In this manner, different promoters can be integrated at the same position in the genome and their activity compared. In some examples a biologically active variant or fragment of the recombinase is provided.

In some examples, the transfer cassette comprises in the following order: the first recombination site, a first polynucleotide, a promoter operably linked to a third recombination site operably linked to a polynucleotide comprising a selectable marker, and a second recombination site, where the first, the second, and the third recombination sites are dissimilar and non-recombinogenic with respect to one another. This transfer cassette can be generically represented as RSa-S1-P1::RSc::S2-RSb. Following the introduction of the transfer cassette at the target site, the activity of the promoter (P1) can be analyzed. Once the activity of the promoter is characterized, additional transfer cassettes comprising a polynucleotide of interest flanked by the second and the third recombination site can be introduced into the plant. Upon recombination, the expression of the polynucleotide of interest will be regulated by a characterized promoter. Accordingly, plant lines having promoters that achieve the desired expression levels in the desired tissues can be engineered so that desired polynucleotides can be readily inserted downstream of the promoter and operably linked to the promoter and thereby expressed in a predictable manner.

A promoter comprises a region of DNA involved in recognition and binding of RNA polymerase and other proteins to initiate transcription. A plant promoter is a promoter capable of initiating transcription in a plant cell.

In further examples, methods are provided to identify a transcriptional regulatory region in a plant. A transcriptional regulatory region is any cis acting element that modulates the level of an RNA. Such elements include, but are not limited to, a promoter, an element of a promoter, an enhancer, an intron, an insulator, or a terminator region. Thus, the methods find use in generating enhancer or promoter traps. In one example, the reporter or marker gene of the target site is expressed only when it inserts close to (enhancer trap) or within (promoter trap) another gene. The expression pattern of the reporter gene will depend on the enhancer elements of the gene near or in which the reporter gene inserts. In this example, the target site introduced into the plant can comprise a marker gene flanked by dissimilar and non-recombinogenic recombination sites. The marker gene is either not operably linked to a promoter (promoter trap) or the marker gene is operably linked to a promoter that lacks enhancer elements (enhancer trap). Following insertion of the target site into the genome of the plant, the expression pattern of the marker gene is determined for each transformant. When a transformant with an expression pattern of the marker gene of interest is found, the enhancer/promoter trap sequences can be used as a probe to clone the gene that has that expression pattern, or alternatively to identify the promoter or enhancer regulating the expression. In addition, once a target site is integrated and under transcriptional control of a transcriptional regulatory element, the methods can further be employed to introduce a transfer cassette having a polynucleotide of interest into the plant. A recombination event between the target site and the transfer cassette will allow the nucleotide sequence of interest to come under the transcriptional control of the promoter and/or enhancer element. See, for example, Geisler et al. (2002) Plant Physiol 130:1747-1753; Topping et al. (1997) Plant Cell 10:1713-245; Friedrich et al. (1991) Genes Dev 5:1513-23; Dunn et al. (2003) Appl Environ Microbiol 1197-1205; and von Melchner et al. (1992) Genes Dev 6:919-27.

Also provided is a method to identify elements that modulate expression in a plant. The method comprises injecting into the embryo sac a composition comprising an effective concentration of an Agrobacterium comprising a T-DNA comprising a target site comprising a selectable marker, wherein the target site is flanked by a first and a second recombination site, wherein the first and the second recombination sites are dissimilar and non-recombinogenic with respect to one another. The plant having modified expression of the selectable marker, increase or decrease and/or spatial or temporal changes is identified. The regulatory element allowing for the modulated expression of the selectable marker can be then be characterized, for example, cloned and/or sequenced using standard methods known.

In further examples, an embryo sac from a plant having the cis regulatory element modulating transcription of the sequence operably linked to the target site is injected with a composition comprising an effective concentration of an Agrobacterium comprising a T-DNA comprising a transfer cassette comprising in the following order: the first recombination site, a polynucleotide of interest and the second recombination site. A recombinase is provided that recognizes and implements recombination at the first and the second recombination sites. In some examples a biologically active variant or fragment of the recombinase is provided.

In other examples, the target site is constructed to have multiple functional sets of dissimilar and non-recombinogenic recombination sites. Thus, multiple genes or polynucleotides can be stacked at the target site. In specific examples, this allows for the stacking of polynucleotides of interest at a precise location in the genome of a plant. Likewise, once a target site has been established within a plant, additional recombination sites may be introduced by incorporating such sites within the transfer cassette. Thus, once a target site has been established, it is possible to subsequently add sites or alter sites through recombination. Such methods are described in detail in WO 99/25821.

In one example, methods to combine multiple transfer cassettes are provided. The method comprises providing an embryo sac from a plant which has stably incorporated into its genome a target site comprising a first recombination site and a second recombination site, wherein the first and the second recombination sites are dissimilar and non-recombinogenic with respect to one another. The embryo sac is injected with a composition comprising an effective concentration of an Agrobacterium comprising a T-DNA comprising a transfer cassette. The transfer cassette comprises, in the following order, at least the first, a third, and the second functional recombination site, wherein the first and the third recombination sites of the transfer cassette flank the polynucleotide of interest, and the first, the second, and the third recombination sites are dissimilar and non-recombinogenic with respect to one another, wherein the Agrobacterium is capable of T-DNA transfer into a plant cell. A recombinase is provided, and the polynucleotide of interest is inserted at the target site. A targeted plant having the polynucleotide of interest stably integrated into its genome is recovered. In some examples a biologically active variant or fragment of the recombinase is provided.

In further examples, the embryo sac from the targeted plant described above, is injected with a second composition comprising an effective concentration of a second Agrobacterium comprising a second T-DNA comprising a second transfer cassette comprising at least the second and the third recombination site, wherein the second and the third recombination sites of the second transfer cassette flank a second polynucleotide of interest; and, wherein the Agrobacterium is capable of T-DNA transfer into a plant cell. A second recombinase is provided, and the second polynucleotide of interest is inserted at the second and third recombination site of the second transfer cassette. In specific examples, a re-targeted plant having the first and the second polynucleotide of interest stably integrated into its genome is recovered from the targeted embryo sac. In some examples a biologically active variant or fragment of the second recombinase is provided. This method allows multiple polynucleotides of interest to be stacked in a predetermined position of the genome of the plant.

Various alterations can be made to the stacking method described above. For instance, an embryo sac from a plant which has stably incorporated into its genome a target site comprising, in the following order, at least a first, a second, and a third recombination site; wherein the first, the second, and the third recombination sites are dissimilar and non-recombinogenic with respect to one another is provided. The embryo sac is injected with a composition comprising an effective concentration of an Agrobacterium comprising a T-DNA comprising a transfer cassette. The transfer cassette comprises in the following order the first recombination site, the polynucleotide of interest, and the second recombination site, wherein the Agrobacterium is capable of T-DNA transfer into a plant cell. A recombinase is provided, wherein the recombinase recognizes and implements recombination at the first and the second recombination site, and the polynucleotide of interest is inserted at the target site. In some examples a biologically active variant or fragment of the recombinase is provided. A targeted plant having the polynucleotide of interest stably integrated into its genome is recovered. An embryo sac from the targeted plant is injected with a second composition comprising an effective concentration of a second Agrobacterium comprising a second T-DNA comprising a second transfer cassette comprising a second polynucleotide of interest flanked by at least the second and the third recombination sites. A second recombinase is provided, wherein the second recombinase recognizes and implements recombination at the second and third recombination sites. In some examples a biologically active variant or fragment of the second recombinase is provided. In specific examples, a re-targeted plant is recovered from the targeted embryo sac.

In some examples, multiple cassettes are stacked, and/or a target site re-targeted, using an embryo sac injection method in combination with any other method for providing a transfer cassette, and/or a recombinase, including particle bombardment, Agrobacterium-mediated transformation, protoplast transformation, electroporation, and the like. For example, if the first transfer cassette is provided by microinjection of an Agrobacterium comprising a T-DNA comprising the transfer cassette, a second transfer cassette can be provided by microinjection DNA, RNA, plasmid, linear polynucleotide, Agrobacterium comprising the second transfer cassette, particle bombardment, Agrobacterium-mediated transformation, or any other known plant transformation method.

In other examples, methods are provided to minimize or eliminate expression resulting from random integration of DNA sequences into the genome a plant. This method comprises providing an embryo sac from the plant, wherein the plant has stably incorporated into its genome a polynucleotide comprising, in the following order, a promoter active in the plant operably linked to an ATG translational start site operably linked to the target site comprising the first recombination site and the second recombination site, wherein the first and the second recombination sites are dissimilar and non-recombinogenic with respect to one another. The embryo sac is injected with a composition comprising an effective concentration of an Agrobacterium comprising a T-DNA comprising a transfer cassette comprising in the following order the first recombination site, the polynucleotide sequence of interest, and the second recombination site. The ATG translation start of the polynucleotide sequence of interest has been replaced with the first recombination site. A recombinase is provided, and the polynucleotide of interest is inserted at the target site. Recombination with the target site results in the polynucleotide of interest being operably linked to the ATG translational start site of the target site. The linkage between a translational start site and a promoter and/or a recombination site generates an inframe fusion that results in a properly expressed and functional gene product. In specific examples, a targeted plant having the polynucleotide of interest stably integrated into the genome is recovered from the embryo sac. In some examples a biologically active variant or fragment of the recombinase is provided.

As a non-limiting example, a transfer cassette comprising RSc::S3(noATG)-RSd, where RS represents a recombination site and S represents a polynucleotide of interest, is injected into an embryo sac of a plant which has stably incorporated into its genome a polynucleotide comprising P1-RSa-S1-T1-RSb-P2-ATG::RSc-S2(no ATG)-T2-RSd, where P represents a promoter, T represents a terminator, RS represents a recombination site, and the symbol::indicates an operable link between adjacent elements. An appropriate recombinase is provided and recombination takes place at the recombination sites such that the sequence between the recombination sites of the transfer cassette replaces the sequence between the corresponding recombination sites of the target site, thereby yielding a directionally targeted and reintegrated new sequence. The new gene (S3) is now driven off of the P2 promoter in the target site. Designing some of the constructs without an ATG start codon on the nucleotide sequence of interest minimizes the probability of expression of the sequence of interest by random integration events, since in order for this to happen, the transfer cassette would need to integrate behind an endogenous promoter region and in the correct reading frame. In some examples a biologically active variant or fragment of the recombinase is provided.

In another example, a plurality of copies of the polynucleotide of interest is provided to the plant. This approach may be accomplished by the incorporation of an extrachromosomal replicon into the transfer cassette, or incorporation of a transfer cassette into the extrachromosomal replicon. Such a method is described in detail in WO 99/25855. In specific examples, the transfer cassette comprises a replicon and a polynucleotide of interest flanked by a directly repeated first and second recombination site, wherein the recombination sites are recombinogenic with respect to one another. When an appropriate recombinase is provided, the transfer cassette flanked by the directly repeated first and second recombination sites is excised from the genome of the plant, producing a viable replicon containing the polynucleotide of interest. Replication should generate a high number of copies of the replicon, and the polynucleotide of interest and may also prolong the availability of the transfer cassette within the cell. In other examples, a third recombination site is present between the replicon and the polynucleotide of interest, wherein the third and the first recombination sites are dissimilar and non-recombinogenic with respect to one another, and the presence of the appropriate recombinase allows integration of the polynucleotide of interest into a target site flanked by the third and the first recombination sites.

A replicon comprises an extrachromosomal self-replicating unit. The replicon can originate from a virus, plasmid or cell and has the capacity for self-replication. In this example, the transfer cassette comprises both a replicon and the polynucleotide of interest. In one example, an organism having a target site stably incorporated into its genome is provided. A transfer cassette comprising in a 5′ to 3′ or 3′ to 5′ orientation: a first recombination site, a replicon, a second recombination site, the polynucleotide of interest, and a third recombination site is provided. The first and third recombination sites of this transfer cassette are directly repeated, corresponding and recombinogenic with respect to each, and the second recombination site is dissimilar and non-recombinogenic with respect to the first and the third recombination sites. The transfer cassette can be contained in a T-DNA.

In one example, the replicon is a viral replicon. An excised viral DNA is capable of acting as a replicon or replication intermediate, either independently, or with factors supplied in trans. The viral DNA may or may not encode infectious viral particles and furthermore may contain insertions, deletions, substitutions, rearrangements or other modifications. The viral DNA may contain heterologous DNA. In this case, heterologous DNA refers to any non-viral DNA or DNA from a different virus. For example, the heterologous DNA may comprise an expression cassette for a protein or RNA of interest.

Viral replicons suitable for use include those from geminivirus, such as begomovirus, curtovirus, or mastrevirus. Viral replicons can also include those of viruses having a circular DNA genome or replication intermediate, such as: Abuitilon mosaic virus (AbMV), African cassava mosaic virus (ACMV), banana streak virus (BSV), bean dwarf mosaic (BDMV), bean golden mosaic virus (BGMV), beet curly top virus (BCTV), beet western yellow virus (BWYV) and other luteoviruses, cassava latent virus (CLV), carnation etched virus (CERV), cauliflower mosaic virus (CaMV), chloris striate mosaic virus (CSMV), commelina yellow mottle virus (CoYMV), cucumber mosaic virus (CMV), dahlia mosaic virus (DaMV), digitaria streak virus (DSV), figwort mosaic virus (FMV), hop stunt viroid (HSV), maize streak virus (MSV), mirabilias mosaic virus (MMV), miscanthus streak virus (MiSV), potato virus X (PVX), potato stunt tuber virus (PSTV), panicum streak virus (PSV), potato yellow mosaic virus (PYMV), rice tungro bacilliform virus (RTBV), soybean chlorotic mottle virus (SoyCMV), squash leaf curl virus (SqLCV), strawberry vein banding virus (SVBV), sugarcane streak virus (SSV), thistle mottle virus (ThMV), tobacco mosaic virus (TMV), tomato golden mosaic virus (TGMV), tomato mottle virus (TMoV), tobacco ringspot virus (TobRV), tobacco yellow dwarf virus (TobYDV), tomato leaf curl virus (TLCV), tomato yellow leaf curl virus (TYLCV), tomato yellow leaf curl virus-Thailand (TYLCV-t) and wheat dwarf virus (WDV) and derivatives thereof. In some examples, the viral replicon may be from MSV, WDV, TGMV or TMV.

In other examples, the insertion of a polynucleotide of interest into the genome of the organism occurs via a single cross over event. For instance, the transfer cassette can comprise a first recombination site, a replicon, a polynucleotide of interest, and a second recombination site. The first and second recombination sites of the transfer cassette are recombinogenic (dissimilar or corresponding) and directly repeated with respect to one another. The target site can comprise a single recombination site that is recombinogenic to one of the recombination sites of the transfer cassette. Such recombinogenic recombination sites can be designed such that integrative recombination events are favored over the excision reaction. Such recombinogenic recombination sites are known and include FLP/FRT, Cre/LOX, and Int/att systems. For example, Albert et al. introduced nucleotide changes into the left 13bp element (LE mutant lox site) or the right 13 bp element (RE mutant lox site) of the lox site. Recombination between the LE mutant lox site and the RE mutant lox site produces the wild-type loxP site and a LE+RE mutant site that is poorly recognized by the recombinase Cre, resulting in a stable integration event (Albert et al. (1995) Plant J. 7:649-659). See also, for example, Araki et al. (1997) Nucleic Acids Res 25:868-872.

The transfer cassette is introduced into the plant comprising the target site. When an appropriate recombinase is provided, a recombination event between the recombinogenic recombination sites of the transfer cassette occurs. This event results in excision of the replicon. Replication of the replicon unit results in a high copy number of the replicon in the organism and may prolong the availability of the transfer cassette in the cell. A second recombination event between the recombinogenic recombination site of the target site and transfer cassette allows the stable integration of the replicon unit and the polynucleotide of interest at the target site of the organism.

Additional methods are provided for locating preferred integration sites within the genome of a plant. The method comprises injecting into an embryo sac a composition comprising an effective concentration of an Agrobacterium comprising a T-DNA comprising a first recombination site and a polynucleotide of interest, wherein the Agrobacterium is capable of T-DNA transfer into a plant cell. In further examples, the T-DNA comprises a target site comprising the first recombination site and a second recombination site, where the first and the second recombination sites are dissimilar and non-recombinogenic with respect to one another. The level of expression of the polynucleotide of interest is determined and an embryo sac or a plant expressing the polynucleotide of interest is selected. The plant harboring this DNA construct can then be characterized for site-specific integration potential, agronomic potential, and copy number.

In further examples, the plant having the preferred integration site is employed to allow for the targeted insertion of other polynucleotides of interest. For instance, once the plant line having the preferred integration site is characterized, additional polynucleotides of interest can be targeted to the integration site using the various methods disclosed herein. For example, an embryo sac having a preferred integration site can be injected with a composition comprising an effective concentration of an Agrobacterium comprising a second T-DNA comprising a transfer cassette, where the transfer cassette comprises, in the following order, the first recombination site, a second polynucleotide of interest, and the second recombination site. A recombinase can be provided and the polynucleotide of interest is inserted at the first recombination site. In some examples a biologically active variant or fragment of the recombinase is provided.

In further examples, methods of providing a recombinase polypeptide to a plant are provided, comprising injecting into an embryo sac of a plant a composition comprising an Agrobacterium comprising a T-DNA comprising a polynucleotide encoding the recombinase operably linked to a promoter active in the embryo sac, wherein the Agrobacterium is capable of T-DNA transfer into a plant cell. In specific examples, the polynucleotide encoding the recombinase is operably linked to a promoter active in the embryo sac. In other examples, the recombinase can be provided as a circular or linear DNA, an RNA, or a polypeptide. In some examples a biologically active variant or fragment of the recombinase polypeptide and/or polynucleotide is provided.

Providing the recombinase in this manner allows various manipulations of integration sites in the plant genome. In one such method, polynucleotide sequences of interest are excised from the plant. For example, the recombinase is provided to a plant having stably incorporated into its genome a nucleotide sequences comprising, in the following order, a first recombination site, a polynucleotide sequence of interest, and a second recombination site, wherein the first and the second recombination sites are recombinogenic with respect to one another and are directly repeated. The recombinase recognizes and implements recombination at the first and the second recombination site, thereby excising the polynucleotide sequence of interest. In another method, the first and the second recombination sites are in an inverted orientation. Upon providing the recombinase, the polynucleotide of interest is inverted.

In further examples, providing the recombinase in this manner further allows the exchange of a transfer cassette and a target site. In one example, the plant has stably incorporated into its genome a target site and a transfer cassette, the target site comprises a first recombination site and a second recombination site. The first and the second recombination sites are dissimilar and non-recombinogenic with respect to one another. The transfer cassette comprises, in the following order, the first recombination site, the polynucleotide of interest, and the second recombination site; wherein the recombinase recognizes and implements recombination at the first and the second recombination site, and the polynucleotide of interest is inserted at the target site.

Polynucleotides can comprise deoxyribonucleotides, ribonucleotides, and any combination of ribonucleotides and deoxyribonucleotides including naturally occurring molecules, modified and/or synthetic analogues. The polynucleotides also encompass all forms of molecules including, but not limited to, single-stranded forms, double-stranded forms, hairpins, stem-and-loop structures, and the like.

The polynucleotide of interest can be provided in an expression cassette for expression in the plant of interest. The cassette may include 5′ and 3′ regulatory sequences operably linked to a polynucleotide of interest. Operably linked indicates a functional linkage between two or more elements. For example, an operable linkage between a polynucleotide of interest a promoter allows expression of the polynucleotide of interest. Operably linked elements may be contiguous or non-contiguous. When used to refer to the joining of two protein coding regions, operably linked indicates that the coding regions are in the same reading frame. The cassette may additionally contain at least one additional gene to be cotransformed into the organism. Alternatively, additional gene(s) can be provided on multiple expression cassettes. The expression cassette may be provided with a plurality of restriction sites and/or recombination sites for insertion of the polynucleotide of interest under the transcriptional regulation of the regulatory regions. The expression cassette may additionally contain selectable marker genes.

The expression cassette can include in the 5′-3′ direction of transcription, a transcriptional and translational initiation region, a polynucleotide of interest, and/or a transcriptional and translational termination region functional in plants. The regulatory regions and/or the polynucleotide of interest may be native/analogous to the host cell and/or to each other. Alternatively, the regulatory regions and/or the polynucleotide of interest may be heterologous to the host cell and/or to each other. Heterologous indicates a sequence that originates from a foreign species, or, if from the same species, is substantially modified from its native form in composition and/or genomic locus by deliberate human intervention. For example, a promoter operably linked to a heterologous polynucleotide may be from a species different from the species from which the polynucleotide was derived, or, if from the same/analogous species, one or both are substantially modified from their original form and/or genomic locus, or the promoter is not the native promoter for the operably linked polynucleotide. A chimeric gene comprises a coding sequence operably linked to a transcription initiation region that is heterologous to the coding sequence. The polynucleotide of interest may be expressed using heterologous promoters, or the native promoter sequences may be used. Such constructs can change expression levels of a polynucleotide of interest in the plant or plant cell. Thus, the phenotype of the plant or plant cell can be altered.

The termination region may be native with the transcriptional initiation region, may be native with the operably linked polynucleotide of interest, may be native with the plant host, or may be derived from another source than the promoter, the polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be modified for increased expression in the transformed plant. For example, the polynucleotides can be synthesized using plant-preferred codons for improved expression. See, for example, Campbell and Gowri (1990) Plant Physiol. 92:1-11 for a discussion of host-preferred codon usage. Methods for synthesizing plant-preferred genes include, for example, U.S. Pat. Nos. 5,380,831, and 5,436,391, and Murray et al. (1989) Nucleic Acids Res. 17:477-498.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. The sequence may also be modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus), (Allison et al. (1986) Virology 154:9-20; and Kong et al. (1998) Arch. Virol. 143:1791-1799), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated to provide the DNA sequences in the proper orientation and, as appropriate, in the proper reading frame. Adapters or linkers may be employed to join the DNA fragments and/or other manipulations may be involved to provide for convenient restriction sites, removal of superfluous DNA, removal of restriction sites, or the like. For this purpose, in vitro mutagenesis, primer repair, restriction, annealing, resubstitutions, transitions, and transversions, may be involved.

A number of promoters can be used. For example, the polynucleotide(s) of interest can be combined with constitutive, tissue-preferred, or any other promoters for expression in plants. For a review of promoters useful in plants see, for example, Potenza et al. (2004) In Vitro Cell Dev Biol 40:1-22.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632; and Christensen et al. (1992) Plant Mol. Biol. 18:675-689); PEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Pathogen-inducible promoters induced following infection by a pathogen include but are not limited to those regulating expression of PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; Van Loon (1985) Plant Mol. Virol. 4:111-116; WO 99/43819; Marineau et al. (1987) Plant Mol. Biol. 9:335-342; Matton et al. (1989) Mol Plant Microbe Interact 2:325-331; Somsisch et al. (1986) Proc. Natl. Acad. Sci. USA 83:2427-2430; Somsisch et al. (1988) Mol. Gen. Genet. 2:93-98; Yang (1996) Proc. Natl. Acad. Sci. USA 93:14972-14977; Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein.

Wound-inducible promoters include potato proteinase inhibitor (pin II) gene (Ryan (1990) Ann. Rev. Phytopath. 28:425-449; Duan et al. (1996) Nat Biotechnol 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792); Eckelkamp et al. (1993) FEBS Lett 323:73-76); MPI gene (Corderok et al. (1994) Plant J. 6(2):141-150); and the like.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize In2-2 promoter, activated by benzenesulfonamide herbicide safeners; the maize GST promoter, activated by hydrophobic electrophilic compounds used as pre-emergent herbicides; and the tobacco PR-1a promoter, activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425, and McNellis et al. (1998) Plant J. 14:247-257); and tetracycline-inducible and tetracycline-repressible promoters (see, for example, Gatz et al. (1991) Mol. Gen. Genet. 227:229-237, and U.S. Pat. Nos. 5,814,618 and 5,789,156).

Tissue-preferred promoters can be utilized to target enhanced expression of a polynucleotide of interest within a particular plant tissue. Tissue-preferred promoters include Kawamata et al. (1997) Plant Cell Physiol. 38:792-803; Hansen et al. (1997) Mol. Gen Genet. 254:337-343; Russell et al. (1997) Transgenic Res. 6:157-168; Rinehart et al. (1996) Plant Physiol. 112:1331-1341; Van Camp et al. (1996) Plant Physiol. 112:525-535; Canevascini et al. (1996) Plant Physiol. 112:513-524; Lam (1994) Results Probl. Cell Differ. 20:181-196; and Guevara-Garcia et al. (1993) Plant J. 4:495-505.

Leaf-preferred promoters are known and include, for example, Yamamoto et al. (1997) Plant J. 12:255-265; Kwon et al. (1994) Plant Physiol. 105:357-67; Yamamoto et al. (1994) Plant Cell Physiol. 35:773-778; Gotor et al. (1993) Plant J. 3:509-18; Orozco et al. (1993) Plant Mol. Biol. 23:1129-1138; and Matsuoka et al. (1993) Proc. Natl. Acad. Sci. USA 90:9586-9590.

Root-preferred promoters are known and include for example, Hire et al. (1992) Plant Mol. Biol. 20:207-218 (soybean root-specific glutamine synthetase gene); Miao et al. (1991) Plant Cell 3:11-22 (cytosolic glutamine synthetase (GS), expressed in roots and root nodules of soybean); Keller and Baumgartner (1991) Plant Cell 3:1051-1061 (root-specific control element in the GRP 1.8 gene of French bean); Sanger et al. (1990) Plant Mol. Biol. 14:433-443 (root-specific promoter of the man nopine synthase (MAS) gene of Agrobacterium tumefaciens); Bogusz et al. (1990) Plant Cell 2:633-641 (root-specific promoters isolated from Parasponia andersonii and Trema tomentosa); Leach and Aoyagi (1991) Plant Sci. 79:69-76 (rolC and rolD root-inducing genes of A. rhizogenes); Teeri et al. (1989) EMBO J. 8:343-350 (wound-induced TR1′ and TR2′ genes); VfENOD-GRP3 gene promoter (Kuster et al. (1995) Plant Mol. Biol. 29:759-772); and rolB promoter (Capana et al. (1994) Plant Mol. Biol. 25:681-691). See also U.S. Pat. Nos. 5,837,876; 5,750,386; 5,633,363; 5,459,252; 5,401,836; 5,110,732; and 5,023,179.

Seed-preferred promoters include both seed-specific promoters active during seed development as well as seed-germinating promoters active during seed germination. See Thompson et al. (1989) BioEssays 10:108. Seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529). For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, gamma-zein, waxy, shrunken 1, shrunken 2, globulin 1, etc. See also WO 00/12733, where seed-preferred promoters from end1 and end2 genes are disclosed.

The expression cassette can also comprise a screenable marker gene. Screenable marker genes are utilized for the identification and/or selection of transformed cells or tissues, and include markers that confer resistance or sensitivity to a compound, visual/screenable markers, and the like. Any marker can be used including genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional markers include phenotypic markers such as β-galactosidase, β-glucuronidase GUS, and fluorescent proteins such as green fluorescent protein (GFP) (Su et al. (2004) Biotechnol Bioeng 85:610-9 and Fetter et al. (2004) Plant Cell 16:215-28), cyan fluorescent protein (CFP) (Bolte et al. (2004) J. Cell Science 117:943-54 and Kato et al. (2002) Plant Physiol 129:913-42), red fluorescent protein, and yellow fluorescent protein (PhiYFP™ from Evrogen, see, Bolte et al. (2004) J. Cell Science 117:943-54). For additional markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724.

The methods can be used for modulating in a plant the concentration and/or activity of the polynucleotide of interest, and in some examples, the polypeptide it encodes. In general, concentration and/or activity is increased or decreased by at least 1%, 5%, 10%, 20%, 30%, 40%, 50%, 60%, 70%, 80%, or 90% relative to a native control plant, plant part, or cell which did not have the polynucleotide of interest introduced. In some examples, concentration and/or activity is increased less than about 1-fold, 1-fold (1×), 2×, 3×, 5×, 7×, 10×, or greater than 10×. Modulation may occur during and/or subsequent to growth of the plant to the desired stage of development. In specific examples, the polynucleotides of interest are modulated in monocots, for example maize.

The expression level of the polynucleotide of interest may be measured directly, for example, by assaying for the level of the polynucleotide or the protein encoded thereby in the plant, or indirectly, for example, by measuring the activity of the polypeptide in the plant.

Methods for inhibiting or eliminating the expression of a gene in a plant are known. Reduction of the activity of specific genes may be desirable for several aspects of genetic engineering in plants. Many techniques for gene silencing are known, including, but not limited to, antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; patent publications US20030175965; US20030180945; WO 99/53050; WO 02/00904; WO 98/53083; Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. (2003) BMC Biotechnology 3:7; Panstruga et al. (2003) Mol. Biol. Rep. 30:135-140; Wesley et al. (2001) Plant J. 27:581-590; and Wang and Waterhouse (2001) Curr. Opin. Plant Biol. 5:146-150; ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); and other methods or combinations of methods known. Any sequence that can decrease the expression of a target polynucleotide can be used, optionally in a T-DNA of an Agrobacterium microinjected into the embryo sac, such as a sequence that produces a miRNA, a sRNA, an antisense RNA, etc., or any combination thereof.

The methods can be employed to provide any polynucleotide of interest, or combination of polynucleotides and/or polypeptides, to the plant. Various changes of interest include modifying the fatty acid composition in a plant, altering the amino acid content of a plant, altering a plant's pathogen defense mechanism, and the like. These results can be achieved by providing expression of heterologous products, or increased expression of endogenous products in plants. Alternatively, the results can be achieved by providing for a reduction of expression of one or more endogenous products, for example, enzymes or cofactors in the plant. These changes may or may not result in a change in phenotype of the transformed plant, a plant part or tissue, and/or transformed seed or part thereof.

General categories of polynucleotides of interest include, for example, those genes involved in information, such as zinc fingers, those involved in communication, such as kinases, and those involved in housekeeping, such as heat shock proteins. More specific categories of transgenes, for example, include genes encoding traits for agronomics, insect resistance, disease resistance, herbicide resistance, sterility, grain characteristics, and commercial products. Polynucleotides of interest include, generally, those involved in oil, starch, protein, carbohydrate, or nutrient metabolism as well as those affecting kernel size, sucrose loading, and the like.

Traits such as oil, starch, and protein content can be genetically altered. Modifications include increasing content of oleic acid, saturated and unsaturated oils, increasing levels of lysine and sulfur, providing essential amino acids, and modification of starch. Hordothionin protein modifications are described in U.S. Pat. Nos. 5,703,049, 5,885,801, 5,885,802, and 5,990,389. Another example is a lysine and/or sulfur rich seed protein encoded by the soybean 2S albumin described in U.S. Pat. No. 5,850,016, and the chymotrypsin inhibitor from barley described in Williamson et al. (1987) Eur. J. Biochem. 165:99-106.

Derivatives of the coding sequences can be made by site-directed mutagenesis to increase the level of preselected amino acids in the encoded polypeptide. For example, the polynucleotides encoding the barley high lysine polypeptide (BHL) are derived from barley chymotrypsin inhibitor, (WO 98/20133). Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502); corn (Pedersen et al. (1986) J. Biol. Chem. 261:6279; Kirihara et al. (1988) Gene 71:359); and rice (Musumura et al. (1989) Plant Mol. Biol. 12:123.

Insect resistance polynucleotides may encode resistance to pests such as rootworm, cutworm, European Corn Borer, and the like. Such polynucleotides include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Polynucleotides encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides, such as chlorosulfuron (e.g., the S4 and/or Hra mutations in ALS), genes coding for resistance to herbicides that act to inhibit action of glutamine synthase, such as phosphinothricin or basta (e.g., the bar gene); glyphosate (e.g., the EPSPS gene and the GAT gene; see, for example, patent publications US20040082770 and WO 03/092360); or other such genes known. Antibiotic resistance can also be provided, for example the nptII gene encodes resistance to the antibiotics kanamycin and geneticin.

Sterility polynucleotides can also be encoded in an expression cassette and provide an alternative to physical detasseling. Examples of polynucleotides used in such ways include male tissue-preferred genes and genes with male sterility phenotypes such as QM, described in U.S. Pat. No. 5,583,210. Other polynucleotides include kinases and those encoding compounds toxic to either male or female gametophytic development.

Commercial traits can also be encoded on a gene or genes that could increase, for example, starch for ethanol production, or provide expression of proteins. Another commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like.

### Experimental

### EXAMPLE 1

### The Targeted Insertion of a Polynucleotide of Interest into the Maize Genome

### A. Generation of DNA Constructs

The DNA constructs are generated using standard molecular biology techniques. See, for example, Molecular Cloning: A Laboratory Manual 2^{nd} ed. (1989) Cold Spring Harbor Laboratory Press, herein incorporated by reference. The DNA construct is then inserted into the desired transformation vectors.

### 1. DNA Construct Comprising the Target Site.

A DNA construct is generated comprising the target site and is used in plant transformations to establish the acceptor plant line. The target site DNA construct PHP17797 was generated for Agrobacterium-mediated transformation: RB—ubiquitin promoter::ubiquitin 5′UTR::ubiquitin intron::FRT1::FLPm::pin II terminator::35SCaMV enhancer::35ScaMV promoter::ADH1 intron::BAR::pin II terminator::FRT5-LB

Plant cells transformed with this target site construct will express FLPm recombinase, and Bar. Bar expression will confer Bialophos resistance.

Vectors comprising an excision activated marker were also created:

PHP10006 Ubi::FRT1::GFP::35S::moPAT::FRT1::GUS::FRT5; and

PHP5869 CAMV35S enh::CAMV35S pro::adh1 intron::(ATG)::FRT1::bar::pinII+space+(TGA)::FRT1::gus::pinII

### 2. DNA Construct Comprising the Transfer Cassette.

A DNA construct is generated comprising the transfer cassette and is inserted into the T-DNA of an Agrobacterium binary vector as described by Bevan et al. (1984) Nucleic Acids Res 12:8711-8721. The transfer cassette DNA construct was generated and inserted into an Agrobacterium vector to generate construct PHP19606:

RB-FRT1::Ubiquitin intron::AmCyan::pinII terminator::FRT5-LB

Recombination of the target site from PHP17797 with the transfer cassette will replace FLPm and BAR, operably linking the cyan fluorescent protein (CFP) coding sequence with the ubiquitin promoter.

### 3. DNA Construct to Monitor Pollen Delivery and/or Fertilization.

In order to identify fertilized embryo sacs, transgenic pollen donor lines were generated comprising visual marker genes. The following DNA constructs were generated:

PHP18098 Ubi promoter::ubiquitin 5′ UTR::ubiquitin intron::AmCyan::pinII terminator; and

PHP18096 Ubi promoter::ZsYellow::pinII terminator.

Embryo sacs from ears fertilized with pollen from a plant transformed with one of the above constructs can be identified by the presence of cyan fluorescent protein (AmCyan) or a yellow fluorescent protein (ZsYellow) in the embryo sac and/or the pollen tube visible in the nucellar slab.

### B. Generation of Maize Target Plants

Transgenic acceptor plant lines can be established via any suitable available transformation method. Examples of transformation via Agrobacterium, particle bombardment, or microinjection are provided below.

I. Transformation and Regeneration of Transgenic Acceptor Plants by Agrobacterium-Mediated Transformation

The DNA construct comprising the target site will be inserted into the T-DNA of an Agrobacterium binary vector as described by Bevan et al. (1984) Nucleic Acids Res 12:8711-8721.

Agrobacterium-mediated transformation of maize is performed essentially as described by Zhao (WO98/32326). Briefly, immature embryos are isolated from maize and the embryos contacted with a suspension of Agrobacterium containing a T-DNA, where the bacteria are capable of transferring the nucleotide sequence of interest to at least one cell of at least one of the immature embryos.

Step 1: Infection Step. In this step the immature embryos are immersed in an Agrobacterium suspension for the initiation of inoculation.

Step 2: Co-cultivation Step. The embryos are co-cultured for a time with the Agrobacterium.

Step 3: Resting Step. Optionally, following co-cultivation, a resting step may be performed. The immature embryos are cultured on solid medium with antibiotic, but without a selecting agent, for elimination of Agrobacterium and for a resting phase for the infected cells.

Step 4: Selection Step. Inoculated embryos are cultured on medium containing a selective agent and growing transformed callus is recovered. The immature embryos are cultured on solid medium with a selective agent resulting in the selective growth of transformed cells.

Step 5: Regeneration Step. Calli grown on selective medium are cultured on solid medium to regenerate the plants.

The target plant is monitored for phenotypic traits associated with both the site specific recombinase and the DNA construct comprising the target site.

II. Transformation and Regeneration of Transgenic Target Plants by Agrobacterium Injection into an Embryo Sac

Nucellar slabs are produced from maize plants at 1 day after pollination, according to the protocols outlined in Laurie et al. (1999) In Vitro Cell Dev Biol Plant 35: 320-325. The pericarp may be removed from each slab prior to injection.

Agrobacterium suspensions are provided and injections are carried out with an Eppendorf FemtoJet microinjector, using pre-pulled glass capillary pipettes (Femtotips II, Eppendorf). For each pipette, the injection pressure was adjusted to allow the smallest possible droplet to be extruded consistently from the tip of the pipette, whilst injection time and compensation pressure are kept at 0.1 s and 50 hpa, respectively.

Both injected and non-injected slabs are cultured on 586P (co-cultivation medium; 586M+100 mM acetosyringone) and maintained in the dark at 26° C. (standard embryo sac culture conditions). Developing embryo-like structures are transferred to hormone-free 272V medium in the light, to allow maturation of shoots and roots. Plantlets were transferred to tubes of 601 G, and subsequently grown to maturity in the greenhouse.

Suspensions of Agrobacterium containing the target site are tested at various concentrations. Injection pressures can range from between about 100 to about 5,000+hpa. In each experiment, droplets of the Agrobacterium suspensions are also expelled from the injection needle onto the surface of 810 medium, before and after injection of the slabs, to check for bacterial growth.

Material is monitored for any effects of injection on subsequent development and plant regeneration, and for evidence of marker expression. If a GUS marker is used, cultures can be stained for GUS as early as a few days after microinjection. Stable transformants can be identified in cultures by straining for GUS expression at about 4-5 weeks after injection.

III. Transformation and Regeneration of Transgenic Target Plants By Bombardment

Immature maize embryos from greenhouse donor plants are bombarded with a plasmid comprising a DNA construct comprising the target site (as described in Example 1A). The plasmid may also contain the selectable marker gene PAT (Wohlleben et al. (1988) Gene 70:25-37) that confers resistance to the herbicide Bialaphos. Transformation is performed as follows.

Preparation of Target Tissue: The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA: The plasmid DNA describe above is precipitated onto 1.0 μm (average diameter) tungsten pellets using a CaCl₂ precipitation procedure as follows: 100 μl prepared tungsten particles in water; 10 μl (1 μg) DNA in TrisEDTA buffer (1 μg total); 100 μl 2.5 M CaC1₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 □l 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates of target embryos are bombarded using approximately 0.1 □g of DNA per shot using the Bio-Rad PDS-1000/He device (Bio-Rad Laboratories, Hercules, Calif.) with a rupture pressure of 650 PSI, a vacuum pressure of 27-28 inches of Hg, and a particle flight distance of 8.5 cm. A total of ten aliquots is taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. The target plant is monitored for phenotypic traits associated with both the site specific recombinase and the DNA construct comprising the target site, while the donor plant will be monitored for phenotypic traits associated with the DNA construct comprising the transfer cassette.

IV. Cell Culture and Regeneration Media

Medium 586M comprises 4.3 g/L MS salts (Gibco 11117-074), 5 ml/L MS Vitamins (0.100 g/L nicotinic acid, 0.02 g/L thiamine HCl, 0.10 g/L pyridoxine HCl, and 0.40 g/L glycine brought to volume with D-I H₂O), 0.5 mg/L thiamine HCl, 0.4 g/L asparagine, 0.1 mg/L BAP (6-benzylaminopurine), and 150 g/L sucrose (brought to volume with D-I H₂O following adjustment to pH 5.8 with KOH); and 3 g/L Gelrite (added after bringing to volume with D-I H₂O).

Culture medium (586P) comprises medium 586M+100 mM acetosyringone.

Culture medium (586N) comprises medium 586M+100 mg/L carbenicillin (added after sterilizing the medium and cooling to room temperature).

Medium (810) comprises 950 ml D-I H₂O, 5 g/L yeast extract (DIFCO), 10 g/L peptone (DIFCO), and 5 g/L NaCl (brought to volume with D-I H₂O after adjustment to pH 6.8); and 15 g/L Bacto-Agar (added after bringing to volume with D-I H₂O); and 50 mg/L spectinomycin (added after sterilizing the medium and cooling to room temperature).

Medium (560Y) comprises 4.0 g/L N6 basal salts (SIGMA C-1416), 1.0 ml/L Eriksson's Vitamin Mix (1000× SIGMA-1511), 0.5 mg/L thiamine HCl, 120.0 g/L sucrose, 1.0 mg/L 2,4-D, and 2.88 g/L L-proline (brought to volume with D-I H₂O following adjustment to pH 5.8 with KOH); 2.0 g/L Gelrite (added after bringing to volume with D-I H₂O); and 8.5 mg/L silver nitrate (added after sterilizing the medium and cooling to room temperature).

Medium (560R) comprises 4.0 g/L N6 basal salts (SIGMA C-1416), 1.0 ml/L Eriksson's Vitamin Mix (1000× SIGMA-1511), 0.5 mg/L thiamine HCl, 30.0 g/L sucrose, and 2.0 mg/L 2,4-D (brought to volume with D-I H₂O following adjustment to pH 5.8 with KOH); 3.0 g/L Gelrite (added after bringing to volume with D-I H₂O); and 0.85 mg/L silver nitrate and 3.0 mg/L bialaphos (both added after sterilizing the medium and cooling to room temperature).

Medium (601G) comprises 2.15 g/L MS salts (GIBCO 11117), 5 ml/L MS vitamins stock solution (0.100 g/L nicotinic acid, 0.02 g/L thiamine HCl, 0.10 g/L pyridoxine HCl, and 0.40 g/L glycine brought to volume with polished D-I H₂O), 0.1 g/L myo-inositol, and 40.0 g/L sucrose (brought to volume with polished D-I H₂O after adjusting pH to 5.6), 0.7 ml/L 1 mg/ml stock IBA, 0.3 ml/L 1 mg/ml stock NAA, (brought to volume with D-I H₂O after adjusting pH to 5.8); and 1.5 g/L Gelrite, sterilized and cooled to 60° C.

Medium (288J) comprises 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl, and 0.40 g/L glycine brought to volume with D-I H₂O) (Murashige and Skoog (1962) Physiol. Plant. 15:473), 100 mg/L myo-inositol, 0.5 mg/L zeatin, 60 g/L sucrose, and 1.0 ml/L of 0.1 mM abscisic acid (brought to volume with D-I H₂O after adjusting to pH 5.6); 3.0 g/L Gelrite (added after bringing to volume with D-I H₂O); and 1.0 mg/L indoleacetic acid and 3.0 mg/L bialaphos (added after sterilizing the medium and cooling to 60° C.).

Medium (272V) comprises 4.3 g/L MS salts (GIBCO 11117-074), 5.0 ml/L MS vitamins stock solution (0.100 g/L nicotinic acid, 0.02 g/L thiamine HCl, 0.10 g/L pyridoxine HCl, and 0.40 g/L glycine brought to volume with D-I H₂O), 0.1 g/L myo-inositol, and 40.0 g/L sucrose (brought to volume with D-I H₂O after adjusting pH to 5.6); and 6 g/L bacto-agar (added after bringing to volume with D-I H₂O), sterilized and cooled to 60° C.

### C. Introducing the Transfer Cassette into the Target Plant

The transfer cassette is introduced into the target plant by microinjecting an embryo sac from the target plant with an Agrobacterium harboring a T-DNA comprising the transfer cassette.

Upon expression of the FLP recombinase in the maize target line, a recombination event between the recombination sites of the target site and the transfer cassette will occur. Plants/embryo sacs having this integration event are recovered.

Fertilized N46 corn inbred embryos are generated by crossing a transgenic N46 line with non-transgenic N46, either line can be used as the female. Transgenic lines comprising:

PHP10006 (Ubi::FRT1::GFP::35S::moPAT::FRT1::GUS::FRT5); or

PHP5869 (CAMV35S enh::CAMV35S pro::adh1 intron::(ATG)::FRT1::bar::pinII+space+(TGA)::FRT1::gus::pinII) were employed. The ears were harvested at 1 DAP. Kernels were sectioned with a Vibratome at 250 μM. Pericarps were removed from the slabs. If the embryos are isolated from ears pollinated by a transgenic line comprising a fluorescent marker expressed in pollen, the putative pollinated embryos can be identified by screening for the pollen marker. For example, when pollen donors comprising PHP18098 (Ubi::ubi intron::cyan FP::pinII) were used, the pollen tube was visible in nucellar slabs.

The vectors employed in the Agrobacterium were PHP22462 (actin-GUS) or PHP17797 (Ubi-FRT1::FLPm::35S::Bar::FRT5).

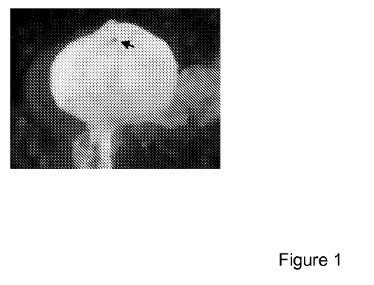

Methods: Agrobacterium containing PHP22462 or PHP17797 were grown on 810 media for 1 day at 28° C. Agrobacterium suspension diluted with 586Q to OD₅₅₀ 0.1 were prepared and injected into the embryo sacs with the microinjector. The following parameters were used with a micropipette type Femtotips II: an injection pressure of 1300 hPa, injection time of 0.1 s, a compensation pressure 50 hPa. After injection, the slabs were cultured on 586P media for 1 to 4 days at 26° C., then transferred to 586N. The results are presented in Table 1 and FIG. 1.

TABLE 1

| | | | # slabs | # slabs |
|---|---|---|---|---|
| | Materials | Vectors | injected | GUS+ |
| Exp #1 | N46-78 × N46 | PHP22462 | 22 | 1 |
| Exp #2 | N46-78 × N46 | PHP22462 | 22 | 2 |
| Exp #3 | N46-78 × N46 | PHP22462 | 20 | 6 |
| Exp #4 | N46 × PHP10006 | PHP17797 | 32 | 1 |

FIG. 1 shows an embryo/endosperm structure derived from a microinjected 1-day post-pollinated slab after 21 days in vitro culture. The nucellar slab isolated from a transgenic plant stably transformed with PHP10006 (Ubi::FRT1::GFP::35S::MOPAT::FRT1::GUS::FRT5) was injected with Agrobacterium containing PHP17797 (RB::Ubi-FRT1::FLPm::35S::Bar::FRT5::LB). The FLP enzyme expressed from PHP17797 mediated excision of the DNA between the FRT1 sites in PHP10006 in the injected slab, thereby creating an operable linkage of the promoterless GUS gene to the Ubi promoter, resulting in GUS expression. The blue spot (arrow) shows the GUS expression in the embryo area, demonstrating T-DNA transfer and gene expression from the injected Agrobacterium. Bar=1 mm.

Optionally, a dye marker is added to the composition comprising the bacterial, or other suspension, with any other components to be injected. For example, FITC-Dextran (Sigma, FD-4), prepared as a 5% w/v solution in distilled water, was mixed 1:1 v/v with the composition for injection. The dextran minimizes diffusion of the dye, allowing visual confirmation of delivery of the composition in nucellar slabs using a fluorescence microscope. No significant plant recovery frequency or developmental differences were observed for embryo sac development from injected material when FITC-Dextran was included in an Agrobacterium suspension.

### EXAMPLE 2

### Microinjection of the Recombinase

The recombinase can be provided in a number of ways. As shown in Example 1, a polynucleotide encoding the recombinase was provided in the DNA construct comprising the target site, or optionally in the transfer cassette. In the target site example, the recombinase is removed from the target site upon recombination with the transfer cassette. In the transfer cassette example, the recombinase may be transiently expressed. Transformants can be screened for integration of the transfer cassette containing the recombinase.

Alternatively, purified FLP protein can be co-injected with the Agrobacterium or a DNA construct comprising the transfer cassette. For example, a 1:1 (by volume) mixture comprising plasmid DNA PHP19606 and FLP protein (0.72 μg/μl) was injected into the one day post-pollination embryo sacs from a corn line comprising an integrated target site from PHP17797. Cyan fluorescence was observed in sectors of calli 22 days after injection, indicating a recombination exchange event between the target site, and the donor plasmid. Fluorescence appeared to be in the cytoplasm of the cells, and the fluorescent callus sectors appeared to be endosperm tissue.

In this example, the target site comprises a polynucleotide encoding FLPm recombinase. Providing supplemental recombinase by co-injection may compensate for any differences between acceptor plant lines in recombinase expression levels and/or recombinase availability. Further, it provides a method to reuse target site once the polynucleotide encoding the recombinase has been removed after the initial recombination exchange reaction.

Alternatively, a DNA construct comprising a polynucleotide encoding the recombinase, operably linked to a promoter, can be co-injected with the transfer cassette. The DNA construct can be in any form that allows transcription and translation of the recombinase, for example in the form of a circular plasmid, or as a linear insert. Further, the DNA construct may be provided alone, or with other factors to facilitate the stability, retention, transcription and/or translation of the DNA construct.

Another option is to co-inject an RNA comprising the recombinase transcript. The RNA transcript can be in any form that allows translation of the recombinase. Further, the RNA may be provided alone, or with other factors to facilitate the stability, retention, and/or translation of the RNA to provide the recombinase.

In some instances, it may be beneficial to provide the recombinase, in any of the forms discussed above, at another time, or in additional times as compared to the injection of the Agrobacterium comprising the transfer cassette. For example, it may be beneficial to provide recombinase by injection before and/or after injecting the Agrobacterium, particularly if the recombinase would be provided in a composition or under conditions which are not optimal for the Agrobacterium.

Upon provision of the FLP recombinase in the maize target line, a recombination event between the recombination sites of the target site and the transfer cassette will occur. Plants/embryo sacs having this integration event are recovered.

Optionally, a dye marker is added to the composition with any other components to be injected. For example, FITC-Dextran (Sigma, FD-4), prepared as a 5% w/v solution in distilled water, is mixed 1:1 v/v with the recombinase composition for injection. The dextran minimizes diffusion of the dye, allowing visual confirmation of delivery of the composition in nucellar slabs using a fluorescence microscope. No significant plant recovery frequency or developmental differences are expected for embryo sac development from injected material when FITC-Dextran is included

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

What is claimed is:

1. A method for targeting a polynucleotide of interest to a target site in a plant comprising
a) providing an embryo sac from the plant, wherein the embryo sac comprises a target site stably incorporated into its genome, the target site comprising a first recombination site;
b) injecting into the embryo sac an effective concentration of an Agrobacterium comprising a T-DNA, wherein the T-DNA comprises the first recombination site and the polynucleotide of interest, wherein the Agrobacterium is capable of T-DNA transfer into a plant cell; and,
c) providing a recombinase, wherein the recombinase recognizes and implements recombination at the first recombination site,
whereby the polynucleotide of interest is inserted at the target site.

2. The method of claim 1 wherein
the embryo sac of step (a) has stably incorporated into its genome the target site, wherein the target site comprises the first recombination site and a second recombination site, wherein the first and the second recombination sites are dissimilar and non-recombinogenic with respect to one another; and,
the T-DNA comprises a transfer cassette, the transfer cassette comprising in the following order, the first recombination site, the polynucleotide of interest, and the second recombination site; and,
wherein the recombinase recognizes and implements recombination at the first and the second recombination sites, whereby the polynucleotide of interest is inserted at the target site.

3. The method of claim 2, wherein the transfer cassette comprises in the following order, the first recombination site, a promoter operably linked to the polynucleotide of interest, and the second recombination site.

4. The method of claim 2 wherein
the embryo sac of step (a) has stably incorporated into its genome a polynucleotide comprising in the following order, a promoter operably linked to the target site,
the transfer cassette comprises in the following order, the first recombination site, the polynucleotide of interest, and the second recombination site, wherein the polynucleotide of interest is not operably linked to a promoter.

5. The method of claim 1, further comprising recovering a targeted plant from the embryo sac, wherein the targeted plant has the polynucleotide of interest stably incorporated into its genome at the target site.

6. The method of claim 2, further comprising recovering a targeted plant from the embryo sac, wherein the targeted plant has the polynucleotide of interest stably incorporated into its genome at the target site.

7. The method of claim 2, wherein
the embryo sac of step (a) has stably incorporated into its genome a polynucleotide comprising in the following order, a promoter active in the plant operably linked to an ATG translational start site operably linked to the target site,
the transfer cassette comprising, in the following order, the first recombination site, the polynucleotide of interest, and the second recombination site, wherein the ATG translation start of the polynucleotide of interest has been replaced with the first recombination site, whereby recombination with the target site results in the polynucleotide of interest being operably linked to the ATG translational start site.

8. The method of claim 1, wherein the providing the recombinase comprises providing a polynucleotide encoding the recombinase, wherein the polynucleotide can be stably integrated in the genome of the plant or the T-DNA.

9. The method of claim 1, wherein the recombinase is a FLP recombinase or a Cre recombinase.

10. The method of claim 8 wherein the recombinase is encoded by a polynucleotide having maize preferred codons.

11. The method of claim 2, wherein the transfer cassette comprises in the following order, the first recombination site, a polynucleotide of interest, a third recombination site, and the second recombination site, wherein the first, the second, and the third recombination sites are dissimilar and non-recombinogenic with respect to one another.

12. The method of claim 2, wherein
the target site comprises in the following order, the first recombination site, the polynucleotide of interest, the second recombination site, and a third recombination site; wherein the first, the second, and the third recombination sites are dissimilar and non-recombinogenic with respect to one another;
the transfer cassette comprises in the following order, the second recombination site, a second polynucleotide of interest, and the third recombination site; and,
providing the recombinase, wherein the recombinase recognizes and implements recombination at the recombination sites of the transfer cassette, whereby the second polynucleotide of interest is inserted at the target site.

13. The method of claim 2, wherein at least one of the dissimilar and non-recombinogenic recombination sites is selected from the group consisting of a FRT site, and a LOX site.

14. The method of claim 1, wherein the first recombination site is selected from the group consisting of a FRT site, and a LOX site.

15. The method of claim 1, wherein the embryo sac comprises a fertilized embryo sac.

16. The method of claim 15, wherein the fertilized embryo sac comprises an embryo or a zygote.

17. The method of claim 1 wherein the plant is a monocot or a dicot.

18. A method of introducing into a plant a recombinase polypeptide comprising injecting into an embryo sac of the plant a composition comprising an Agrobacterium comprising a T-DNA comprising a promoter active in the embryo sac operably linked to a polynucleotide encoding the recombinase, wherein the Agrobacterium is capable of T-DNA transfer into a plant cell.

19. A method for locating preferred integration sites within the genome of a plant comprising
a) injecting into an embryo sac from the plant a composition comprising an effective concentration of an Agrobacterium comprising a T-DNA comprising a first recombination site and a polynucleotide of interest, wherein the Agrobacterium is capable of T-DNA transfer into a plant cell;
b) monitoring the level of expression of the polynucleotide of interest; and,
c) selecting the embryo sac or the plant recovered therefrom expressing the polynucleotide of interest.

20. The method of claim 19 wherein the T-DNA comprises a target site comprising in the following order, the first recombination site, the polynucleotide of interest, and a second recombination site, wherein the first and the second recombination sites are dissimilar and non-recombinogenic with respect to one another.
